(19) United States
(12) Patent Application Publication
(10) Pub. No.: US 2015/0315137 A1
(43) Pub. Date: Nov. 5, 2015

(54) System and Method for Fluoroalkylated Fluorophthalocyanines With Aggregating Properties and Catalytic Driven Pathway for Oxidizing Thiols

(71) Applicants: Sergiu M. Gorun, Montclair, NJ (US); Andrei Ioan Loas, Harrison, NJ (US); Kimberly Griswold, Flanders, NJ (US); Lukasz Lapok, Piekary Slaskie (PL); Hemantbhai Hasmukhbhai Patel, Piscataway, NJ (US); Robert Gerdes, ULM (DE)

(72) Inventors: Sergiu M. Gorun, Montclair, NJ (US); Andrei Ioan Loas, Harrison, NJ (US); Kimberly Griswold, Flanders, NJ (US); Lukasz Lapok, Piekary Slaskie (PL); Hemantbhai Hasmukhbhai Patel, Piscataway, NJ (US); Robert Gerdes, ULM (DE)

(73) Assignee: NEW JERSEY INSTITUTE OF TECHNOLOGY, Newark, NJ (US)

(21) Appl. No.: 14/334,241

(22) Filed: Jul. 17, 2014

Related U.S. Application Data
(63) Continuation of application No. 13/286,393, filed on Nov. 1, 2011, now abandoned.
(60) Provisional application No. 61/409,049, filed on Nov. 1, 2010.
(60) Provisional application No. 61/469,232, filed on Mar. 30, 2011.

(51) Int. Cl. C07C 319/24; B01J 31/18

(52) U.S. Cl. CPC C07C 319/24; B01J 31/183; B01J 2231/70; B01J 2531/842; B01J 2531/845; B01J 2531/22; B01J 2531/26; B01J 2531/16

(57) ABSTRACT

Organo-metallic materials with reduced steric hindrance and the ability to aggregate are disclosed. The metal remains capable of binding additional molecules. As an example, Zn complexes that prove aggregation are provided. Such aggregation may help improve or trigger new surface properties of the materials, alone or in combination with others. In a further implementation of the present disclosure, a robust molecule that resists degradation via nucleophilic, electrophilic and radical attacks is provided. Coordinated O₂ is reduced catalytically, producing efficiently thyil radicals in spite of the extreme electronic deficiency of the catalyst.

### CROSS-REFERENCE TO RELATED APPLICATIONS

The present application is a continuation application that claims the benefit of a co-pending, non-provisional patent application entitled “System and Methods for Fluoroalkylated Fluorophthalocyanines With Aggregating Properties and Catalytic Driven Pathway For Oxidizing Thiols” filed on Nov. 1, 2011, as Ser. No. 13/286,393, and claims the benefit of U.S. Provisional Application Nos. 61/409,049, filed Nov. 1, 2010, and 61/469,232, filed Mar. 30, 2011. The contents of the foregoing patent applications are incorporated herein by reference in their entireties.

### STATEMENT OF GOVERNMENT SUPPORT

The United States government may hold license and/or other rights in this invention as a result of financial support provided by governmental agencies in the development of aspects of the invention. Parts of this work were supported by a grant from the National Science Foundation, Grant No. CBET-0233811, and contracts with the U.S. Army, Contract Nos. DAAE30-03-D-1015-0032 and W15-QKN-10-0503-002.

### BACKGROUND

1. Technical Field

The present invention relates to molecules that lack carbon hydrogen bonds, bind metals and exhibit variable aggregation due to partial steric hindrance. In particular, the present invention relates to fluoroalkylated fluorophthalocyanine molecules, which exhibit novel asymmetry and tunable π-π stacking interactions. The present invention further relates to phthalocyanine molecules that lack carbon hydrogen bonds, bind metals, and broaden the reactivity spectrum of a catalyst while suppressing its nucleophilic, electrophilic and radical degradation pathways.

2. Background Art

Phthalocyanines bearing perfluoroalkyl groups exhibit useful properties, such as surface coverage, coatings and photosensitizing properties. One structural defining property is the presence of perfluoroalkyl groups that impart solubility and variable steric hindrance that precludes the aggregation of the planar phthalocyanine macrocycle via known π-π stacking interactions. Another structural defining property, as depicted in FIG. 1A, is the symmetric characteristic of perfluorophthalocyanines known in the art. The symmetric perfluorophthalocyanines of the prior art thereby exhibit a four-fold axis of rotation.

As shown in FIG. 1A, due to the structural properties of the classical perfluorophthalocyanines, stacking is exhibited both in solution and in the solid state. This stacking characteristic of classical perfluorophthalocyanines severely limits their solubility in organic solvents and, thus, also limits their processability. Such molecules are generally produced via the template tetramerization of various fluorinated precursors, the most common one being the tetrafluorophthalonitrile, as shown in FIG. 2a.

Other exemplary molecules of the prior art are depicted in FIGS. 2A-F. Specifically, FIG. 2A shows tetrafluorophthalonitrile, FIG. 2B shows a F₁₆PcM, a metallo-perfluorophthalocyanine, M=metal ion in the +2 oxidation state, FIG. 2C shows a 4,5-bis(trifluoromethyl)-phthalonitrile (see, e.g., Pawlowski, G. et al., Synthetic Communications, 11, 351 (1981) and Chambers, R. D. et al., Tetrahedron, 54, 4949, (1998)), FIG. 2D shows a metallo-2,3,9,10,16,17,23,24-octakis-(trifluoromethyl)-phthalocyanine, F₂₄H₈PcM (see, e.g., Pawlowski, G. et al., Synthetic Communications, 11, 351 (1981)), FIG. 2E shows a perfluoro-4,5-diisopropyl-phthalonitrile (see, e.g., Gorun, S. M. et al., Journal of Fluorine Chemistry, 91, 37 (1998)), and FIG. 2F shows a metallo-perfluoro-2,3,9,10,16,17,23,24-octakis-(isopropyl)-phthalocyanine, F₆₄PcM (see, e.g., Bench, B. A. et al., Angew. Chem. Int. Ed., 41, 747 (2002) and Bench, B. A. et al., Angew. Chem. Int. Ed., 41, 750 (2002)).

The introduction of iso-perfluoroalkyl groups generally results in the formation of perfluoroalkyl perfluorophthalocyanines that minimize aggregation via an increased degree of steric hindrance. In addition, a significant higher degree of solubility in organic solvents may result. The structural prototype for such molecules is shown in FIGS. 2E-F.

However, a need remains for fluorophthalocyanines which exhibit asymmetric properties and enable stacking, while permitting a high degree of solubility and aggregation.

These and other needs are addressed by the systems and methods of the present disclosure.

### SUMMARY

In accordance with embodiments of the present disclosure, classes of fluoroalkylated fluorophthalocyanine molecules, exhibiting novel asymmetry and tunable π-π stacking interactions are provided. The metal remains capable of binding additional molecules. Such aggregation may help improve or trigger new surface properties of the materials, alone or in combination with others.

In a further implementation of the present disclosure, an organic-based, thermally and chemically robust molecule that may suggest ways to design materials refractory to nucleophilic, electrophilic or radical attack while exhibiting useful aerobic catalytic properties is provided.

Other objects, features and functionalities of the present disclosure will become apparent from the following detailed description considered in conjunction with the accompanying drawings. It is to be understood, however, that the narrative description and drawings are designed as exemplary teachings only and not as a definition of the limits of the present disclosure.

### BRIEF DESCRIPTION OF THE DRAWINGS

To assist those of skill in the art in making and using the disclosed systemsmethods, reference is made to the accompanying figures, wherein:

FIGS. 1A and B illustrate general structures of a) symmetric and b) asymmetric phthalocyanines;

FIGS. 2A-F illustrate prior art molecules, including a) tetrafluorophthalonitrile; b) F₁₆PcM, a metallo-perfluorophthalocyanine, M=metal ion in the +2 oxidation state, c) 4,5-bis(trifluoromethyl)-phthalonitrile, d) metallo-2,3,9,10,16,17,23,24-octakis-(trifluoromethyl)-phthalocyanine, F₂₄H₈PcM, e) perfluoro-4,5-diisopropyl-phthalonitrile, f) metallo-perfluoro-2,3,9,10,16,17,23,24-octakis-(isopropyl)-phthalocyanine, F₆₄PcM;

FIGS. 3A-E depict exemplary classes of molecules described herein, including a) metallo-2,3,9,10,16,17,23,24-octakis-(trifluoromethyl)-tetrafluorophthalocyanine, F₂₈H₄PcM, b) metallo-perfluoro-1,2,4-triisopropyl-phthalocyanine, F₃₄PcM, c) metallo-perfluoro-1,2,4,8,10,11-hexa-isopropyl-phthalocyanine, F₅₂Pc′M, d) metallo-perfluoro-2,3,9,10-tetraisopropyl-phthalocyanine, F₄₀PcM, and e) metallo-perfluoro-2,3,9,10,16,17-hexaisopropyl-phthalocyanine, F₅₂Pc″M;

FIG. 4 depicts an exemplary synthesis scheme pattern for exemplary embodiment F₂₈H₄PcM, with numbering of compounds;

FIGS. 5A-D illustrate X-ray structures of a) 1,2-bis(trifluoromethyl)-3-nitro-4,5-dimethylbenzene, b) 1,2-bis(trifluoromethyl)-3-fluoro-4,5-dimethylbenzene, c) 4,5-bis(trifluoromethyl)-3-fluorophthalic acid, and d) 4,5-bis(trifluoromethyl)-3-fluorophthalonitrile;

FIG. 6 shows the measured exact mass spectrum (positive ion APCI) and isotope pattern of [M+H]⁺ for F₂₈H₄PcZn;

FIGS. 7A-C display UV-Vis data comparison in acetone of partially aggregated b) F₂₈H₄PcZn with sterically non-hindered a) F₁₆PcZn and sterically hindered c) F₆₄PcZn;

FIGS. 8A-D display UV-Vis electronic absorption spectra of F₂₈H₄PcZn, depicting strong solvent-dependent aggregation: a) chloroform, monomer (minimal aggregation); b) ethyl acetate, mostly monomer; c) acetone, intermediate aggregation; d) ethanol, mostly aggregated;

FIGS. 9A-C illustrate a) the X-ray structure of F₂₈H₄PcZn(CH₃CN) showing metal-coordinated acetonitrile, b) the top view of the π-π stacking region of two adjacent molecules of F₂₈H₄PcZn, and c) the side view of the aggregation of F₂₈H₄PcZn in solid state (ball-and-stick representation);

FIG. 10 shows the measured exact mass spectrum (positive ion APCI) and isotope pattern of [M+H]⁺ for F₂₈H₄PcCo;

FIG. 11 depicts an exemplary synthesis scheme for production of asymmetric F₃₄PcM and F₅₂Pc′M, showing the results of the combination of precursors P0 and P3;

FIG. 12 shows the measured exact mass spectrum (positive ion APCI) and isotope pattern of [M+H]⁺ for F₃₄PcZn;

FIGS. 13A and B display the UV-Vis electronic absorption spectra of F₃₄PcZn showing solvent-dependent aggregation: a) chloroform, monomer; b) ethanol, significant degree of dimerization;

FIG. 14 shows the measured exact mass spectrum (positive ion APCI) and isotope pattern of [M+H]⁺ for F₅₂Pc′Zn;

FIG. 15 shows the X-ray structure of F₅₂Pc′Zn(OPPh₃);

FIG. 16 illustrates the aggregation in solid state (side view) of F₅₂Pc′Zn;

FIG. 17 shows the measured exact mass spectrum (negative ion APCI) and isotope pattern of [M]⁻ for F₃₄PcCo;

FIGS. 18A and B illustrate a) the aggregation in solid state (side view) of F₃₄PcCo, and b) a top view of the π-π stacking region of two adjacent molecules of F₃₄PcCo;

FIG. 19 shows the X-ray structure of F₃₄PcCo(CH₃CN);

FIG. 20 shows the measured exact mass spectrum (negative ion APCI) and isotope pattern of [M]⁻ for F₅₂Pc′Co;

FIGS. 21A-D depict a) a ball and-stick representation of F₃₄PcZn(H₂O) ((CH₃)₂CO)₂, b) a van der Waals representation of F₃₄PcZn(H₂O)((CH₃)₂CO)₂, c) aggregation in solid state of F₃₄PcZn(H₂O) (side view), and d) a top view of the π-π stacking region of two adjacent molecules of F₃₄PcZn(H₂O);

FIG. 22 illustrates the X-ray structure of F₃₄PcZn(H₂O);

FIG. 23 illustrates an exemplary synthesis scheme for production of asymmetric F₄₀PcM and F₅₂Pc″M, showing the results of the combination of precursors P0 and P2;

FIG. 24 shows the measured exact mass spectrum (positive ion APCI) and isotope pattern of [M+H]⁺ for F₄₀PcZn;

FIG. 25 illustrates the X-ray structure of F₄₀PcZn(OPPh₃);

FIGS. 26A and B illustrate a) the aggregation in solid state (side view) of F₄₀PcZn(OPPh₃), and b) a top-down view of the π-π stacking region of two adjacent molecules of F₄₀PcZn;

FIGS. 27A and B display the UV-Vis electronic absorption spectra of F₄₀PcZn showing solvent-dependent aggregation: a) chloroform, monomer; b) ethanol, strong aggregation;

FIG. 28 shows the measured exact mass spectrum (positive ion APCI) and isotope pattern of [M+H]⁺ for F₅₂Pc″Zn;

FIG. 29 shows the measured exact mass spectrum (positive ion ESI) and isotope pattern of [M+H]⁺ for F₄₀PcCo;

FIG. 30 illustrates the X-ray structure of F₄₀PcCo(H₂O);

FIG. 31 shows the measured exact mass spectrum (negative ion APCI) and isotope pattern of [M]⁻ for F₅₂Pc″Co;

FIGS. 32A and B display the UV-Vis electronic absorption spectra of F₅₂Pc″Co showing solvent-dependent aggregation: a) chloroform, slightly aggregated; b) tetrahydrofuran, increased degree of aggregation;

FIGS. 33A and B illustrate a) exemplary cobalt phthalocyanines, and b) F₆₄PcCo(O₂) reaction intermediate, drawn based on the X-ray structure of F₆₄PcCo((CH₃)₂CO)₂;

FIGS. 34A and B display a) a plot of Pc(Co(II)Co(I)) reduction potentials vs. the sum of substituents Hammett σ constants, and b) O₂ consumption in the catalyzed autooxidation of 2-mercaptoethanol in aqueous tetrahydrofuran;

FIGS. 35A and B display a) ESR spectrum of F₆₄PcCo in acetone, and b) ESR spectrum of F₆₄PcCo in acetoneN-methyl imidazole;

FIG. 36 illustrates the UV-Vis titration of F₆₄PcCo with aqueous NaOH in THF;

FIG. 37 shows the ratio of catalysts Q-bands intensities after 5 h and 24 h, relative to initial intensities, taken as a measure of catalyst stability, during the autooxidation of 2-mercaptoethanol in aqueous tetrahydrofuran;

FIGS. 38A-C display UV-Vis monitored catalyst stability of a) F₁₆PcCo, b) F₆₄PcCo, and c) H₁₆PcCo during the autooxidation of 2-mercaptoethanol in aqueous tetrahydrofuran; and

FIG. 39 illustrates the O₂ consumption in the catalyzed oxidation of perfluoro benzenethiol, with the inset depicting the parallel reaction of thioether-thiol formation via nucleophilic attack in the absence of the catalyst.

### DESCRIPTION OF EXEMPLARY EMBODIMENT(S)

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

### Fluoroalkylated Fluorophthalocyanines

In accordance with embodiments of the present disclosure, classes of fluoroalkylated fluorophthalocyanine molecules, exhibiting novel asymmetry and tunable π-π stacking interactions are provided. In particular, a composition is disclosed including a phthalocyanine molecule, the phthalocyanine molecule exhibiting an asymmetric orientation and the phthalocyanine molecule exhibiting tunable π-π stacking. The phthalocyanine molecule is generally a fluoroalkylated fluorophthalocyanine molecule, is capable of aggregation and is adapted to form intermolecular interactions. Further, the phthalocyanine molecule may be produced by template tetramerization and exhibits tunable π-π stacking in a solution state and a solid state. The asymmetric orientation of the disclosed phthalocyanine provides advantageous properties, including increased solubility, variability and tenability in aggregation, compatibility with polymers, variable film forming properties, a variable optical property, and tunable magnetic and electronic interactions.

In accordance with embodiments of the present disclosure, a method for forming a composition is also provided. The disclosed method generally involves introducing a phthalocyanine molecule, the phthalocyanine molecule exhibiting an asymmetric orientation and tunable π-π stacking.

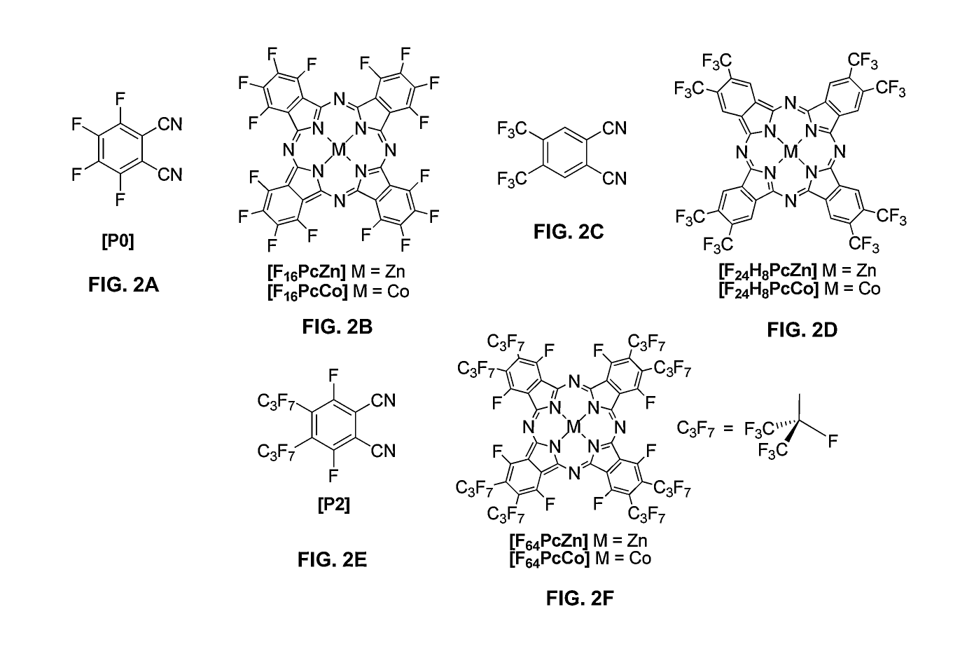

Similar to the case of the F₁₆PcM (Pc=phthalocyanine and M=metal), F₂₄H₈PcM, and F₆₄PcM molecules, depicted in FIGS. 2A-D, the new classes of molecules, F₂₈H₄PcM, F₃₄PcM, F₅₂Pc′M, and F₅₂Pc″M may be produced by template tetramerization.

While advantageous from enhanced thermal and chemical stability points of view, these new classes also form thin films on various surfaces. Such films exhibit physical and chemical properties that depend on the chemical composition of the phthalocyanine, including the ability to form intermolecular interactions that presumably would stabilize a derived material with long range order and superior coverage properties. Thus, materials that retain a high degree of fluorination and solubility in organic solvents, while exhibiting intermolecular interactions are desirable. Described herein is the production of exemplary new classes of such materials that exhibit π-π stacking interactions in solution and/or solid state.

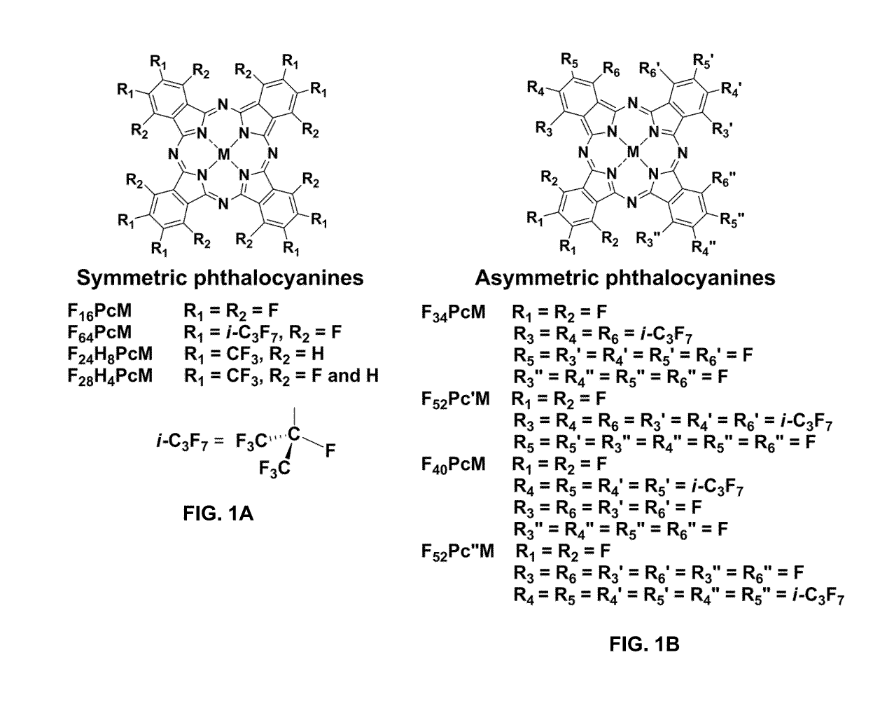

Unlike the F₁₆, F₂₄H₈ and F₆₄PcMs, variants of the exemplary new classes exhibit asymmetric perfluorinated phthalocyanine molecules. FIG. 1B illustrates exemplary general structures of asymmetric phthalocyanines. By asymmetry, it is meant that unlike the F₁₆, F₂₄H₈ and F₆₄ variants, the new classes do not exhibit four-fold axis of rotation. The resulting mirror plane geometry allows for increased solubility and the ability to form partial or total π-π stacking, as well as the advantageous properties of variability and tunability in aggregation, enhanced compatibility with polymers, variable film forming properties, variable optical properties, tunable magnetic and electronic interactions.

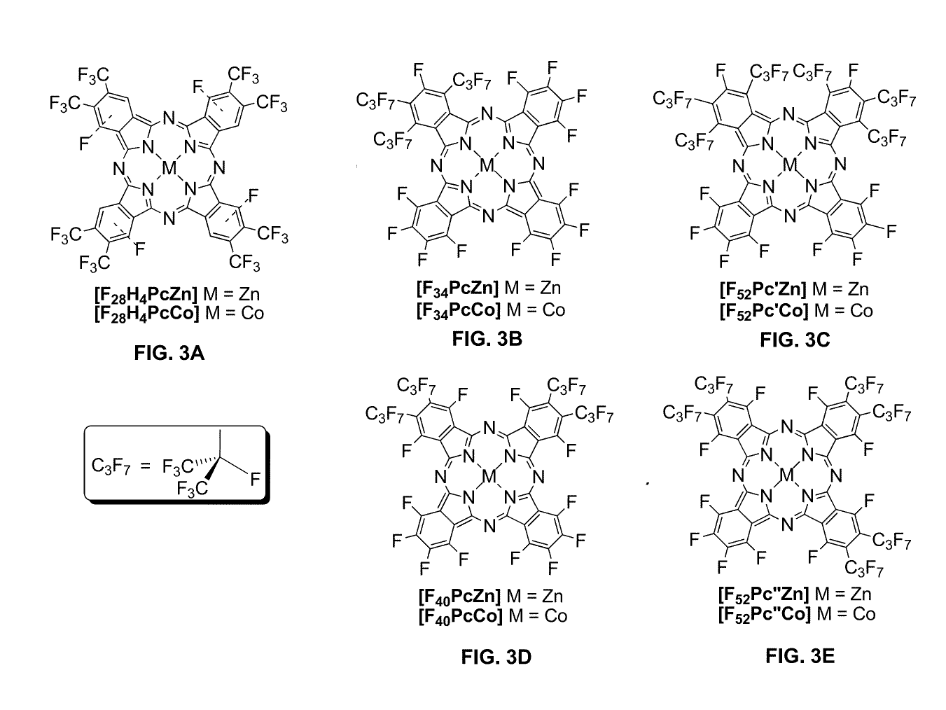

Turning now to FIGS. 3A-E, exemplary classes of molecules described herein are depicted. In particular, FIG. 3A shows a metallo-2,3,9,10,16,17,23,24-octakis-(trifluoromethyl)-tetrafluorophthalocyanine, F₂₈H₄PcM, FIG. 3B shows a metallo-perfluoro-1,2,4-triisopropyl-phthalocyanine, F₃₄PcM, FIG. 3C shows a metallo-perfluoro-1,2,4,8,10,11-hexa-isopropyl-phthalocyanine, F₅₂Pc′M, FIG. 3D shows a metallo-perfluoro-2,3,9,10-tetraisopropyl-phthalocyanine, F₄₀PcM, and FIG. 3E shows a metallo-perfluoro-2,3,9,10,16,17-hexaisopropyl-phthalocyanine, F₅₂Pc″M. The asymmetric structure of the exemplary phthalocyanines, as discussed above with respect to FIG. 1B, can be distinctly seen in FIGS. 3A-E.

The synthesis of all new F₃₄PcM, F₄₀PcM, F₅₂Pc′M, and F₅₂Pc″M complexes has been accomplished by mixing the precursors P0, P2 and/or P3, taken in the appropriate ratios for the desired product with a metal salt, usually acetate. Precursor P0 is generally equivalent to tetrafluorophthalonitrile, as shown in FIG. 2A, precursor P2 is generally equivalent to perfluoro-4,5-diisopropyl-phthalonitrile, as shown in FIG. 2E, and precursor P3 is generally equivalent to perfluoro-3,5,6-triisopropyl phthalonitrile. Heating the mixtures using microwave radiation results in crude products that are subjected to chromatographic separations using silica gel and mixtures of acetone-hexanes with a progressively higher ratio of acetone (approximately 1:10 to 10:1). The yields vary depending on the particular product and whether the chromatography is repeated. Because the above procedure is generally applicable for all metals, the experimental models discussed herein are shown for illustrative purposes only and do not limit the scope of the disclosure.

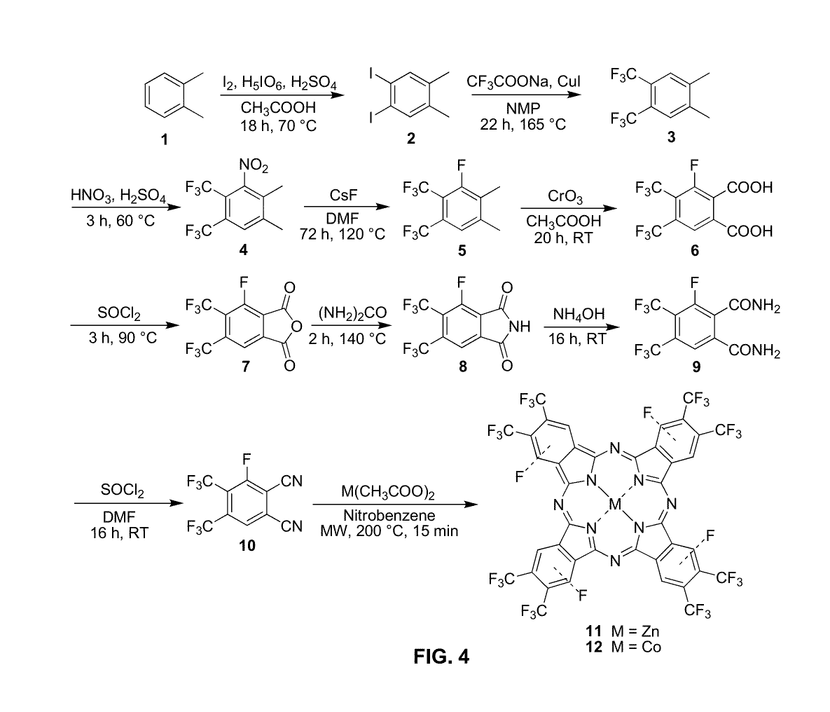

Turning now to FIG. 4, F₂₈H₄PcM complexes are synthesized by the exemplary process depicted, with numbering of compounds. The present invention is not limited to the metals in the experimental exemplary embodiments. The products are best characterized by ¹⁹F NMR, as well as by mass spectrometry. Single-crystal X-ray diffraction further provides both confirmation of compositional identity and also atomic-resolution of molecular and solid-state architectures. The compositional identity of the products is unambiguously established by mass spectrometry.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### Example 1

In one exemplary embodiment, F₂₈H₄PcM is produced using the synthesis scheme described in FIG. 4 and the metal used is Zn. As will be apparent to one of ordinary skill in the art, the present exemplary embodiment embraces the use of multiple other metals as the synthesis scheme is not metal specific and would include, but not be limited to, other metals with ionic radii that would be coordinated by the four nitrogen atoms of the phthalocyanines, e.g., Co, Fe, Mg, Cu, and the like.

The exemplary synthesis scheme described in FIG. 4 includes Compounds 1-12, which will be discussed in greater detail below.

### Compounds 1 and 2

With reference to Compounds 1 and 2 of FIG. 4, an exemplary synthesis and characterization of 1,2-diiodo-4,5-dimethylbenzene (hereinafter “Compound 2”) is depicted. In particular, a mixture of o-xylene (about 40.0 g, 0.377 mol), periodic acid (about 34.4 g, 0.151 mol) and iodine (about 84 g, 0.339 mol) is heated under stirring in a solution of acetic acid (about 200 mL), water (about 40 mL) and sulfuric acid 96% (about 6 mL) to approximately 70° C. for about 18 h. After cooling to about room temperature, the reaction mixture is poured over a solution of about 20 g Na₂S₂O₃ in about 400 mL water, and about 300 mL CH₂Cl₂ is added. Intense stirring for approximately five (5) minutes allows for the reduction of iodine. The organic phase is separated and the water phase is washed with CH₂Cl₂ (about 2×150 mL). The combined organic layers are washed with a solution of about 15 g Na₂CO₃ in about 450 mL water (about 3×150 mL), dried over MgSO₄, filtered, evaporated in vacuo and recrystallized from methanol (about 700 mL) to afford white crystalline plates of Compound 2 in about 69% yield (about 83.4 g).

Specifically, the exemplary properties of Compound 2 are as follows: Mp: about 88-90° C. (taught by prior literature as 91° C. (see, e.g., Kovalenko, S. V. et al., Org. Lett., 6(14), 2457 (2004))); ¹H NMR (300 MHz, (CD₃)₂CO): δ 2.17 (6H, s, CH₃), 7.69 (2H, s, Ph-H); ¹³C {¹H} NMR (75 MHz, (CD₃)₂CO) δ 18.9, 104.2, 139.9, 140.8.

### Compound 3

With reference to Compound 3 of FIG. 4, an exemplary synthesis and characterization of 1,2-bis(trifluoromethyl)-4,5-dimethylbenzene (hereinafter “Compound 3”) is depicted. In particular, dry sodium trifluoroacetate (about 21.8 g, 0.16 mol) and copper iodide (about 30.5 g, 0.16 mol) is mixed in about 150 mL dry NMP. To this suspension, a solution of Compound 2 (about 7.2 g, 0.02 mol) in about 50 mL dry NMP is added under stirring at approximately room temperature. The reaction mixture is then heated under nitrogen and kept at about 165° C. for about 22 h. Evolution of CO₂ may be monitored with an oil bubbler. After cooling, the mixture is poured into about 500 mL of hexanes, stirred intensively for about 30 min and allowed to settle. The upper hexane phase is filtered over silica gel, washed with water (about 3×150 mL) and then dried over MgSO₄, filtered off and evaporated under reduced pressure until about 150 mL remain. This solution is further separated by flash chromatography with hexanes over silica gel. The product is collected as the top fraction. Careful removal of the solvent under a nitrogen stream followed by standing in the freezer for approximately 30 min allowed for separation of Compound 3 as colorless crystals in about 72% yield (about 3.5 g).

Specifically, the exemplary properties of Compound 3 are as follows: Mp: 38-39° C. (taught by prior literature as ranging from 38-40° C. (see, e.g., Pawlowski, G. et al., Synthetic Communications, 11, 351 (1981) and Chambers, R. D. et al., Tetrahedron, 54, 4949, (1998))); ¹H NMR (300 MHz, CDCl₃): δ 2.37 (6H, s, CH₃), 7.58 (2H, s, Ph-H); ¹⁹F NMR (282 MHz, CDCl₃): δ 59.58 (6F, s, CF₃).

### Compound 4

With reference to Compound 4 of FIG. 4, an exemplary synthesis and characterization of 1,2-bis(trifluoromethyl)-3-nitro-4,5-dimethylbenzene (hereinafter “Compound 4”) is depicted. In particular, a mixture of about 40 mL sulfuric acid about 96% (about 74 g, 750 mmol) and about 10 mL fuming nitric acid (about 15.2 g, 240 mmol) is given under stirring to Compound 3 (about 4.2 g, 17.2 mmol) and heated to approximately 60° C. for about 3 h. After cooling to about room temperature, the mixture is poured over about 300 g crushed ice. The milky solution is then extracted with CH₂Cl₂ (about 2×100 mL). The combined organic fractions are washed with about 3% Na₂CO₃ solution (about 2×150 mL) and then water (about 2×200 mL). The CH₂Cl₂ solution is dried over MgSO₄, filtered and evaporated in vacuo. The crude yellowish solid is purified via silica gel filtration using hexanes to give white crystals of Compound 4 in about 90% yield (about 4.45 g).

Specifically, the exemplary properties of Compound 4 are as follows: Mp: 45-46° C.; IR (KBr): 3075, 2924, 1620, 1554, 1453, 1380, 1319, 1278, 1156, 1010, 952, 904, 768 cm⁻¹; ¹H NMR (300 MHz, (CD₃)₂CO): δ 2.32 (3H, s, 5-CH₃), 2.61 (3H, s, 4-CH₃), 8.10 (1H, s, Ph-H); ¹⁹F NMR (282 MHz, (CD₃)₂CO): δ 55.25 (3F, s, 2-CF₃), −57.85 (3F, s, 1-CF₃); ¹³C {¹H} NMR (75 MHz, (CD₃)₂CO): δ 14.8 (s), 20.8 (s), 118.1 (q, J_{C—F}=35.0 Hz), 122.6 (q, J_{C—F}=274.5 Hz), 123.5 (q, T_{C—F}=273.4 Hz), 127.3 (q, J_{C—F}=33.8 Hz), 131.5 (q, J_{C—F}=6.2 Hz), 135.4 (s), 147.3 (s), 151.2 (s); HRMS (EI): calcd. for [M]⁺ (C₁₀H₇F₆NO₂)—⁺ 287.0381. found 287.0389.

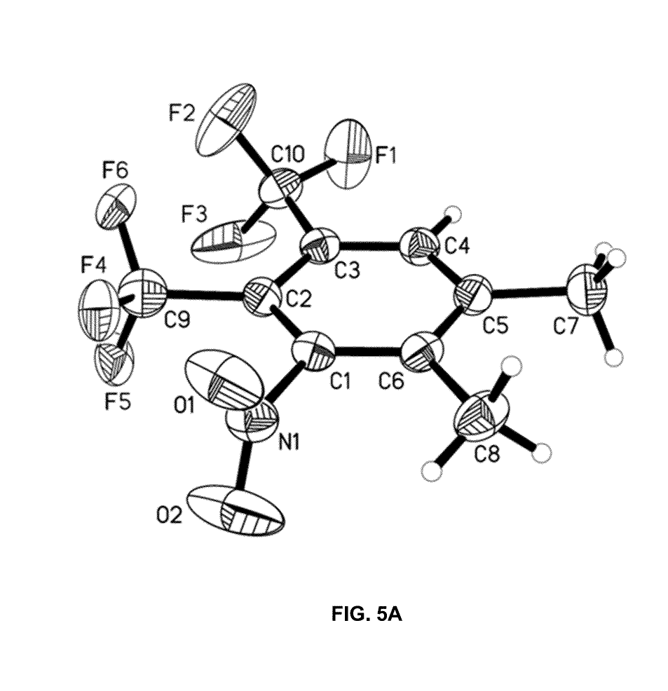

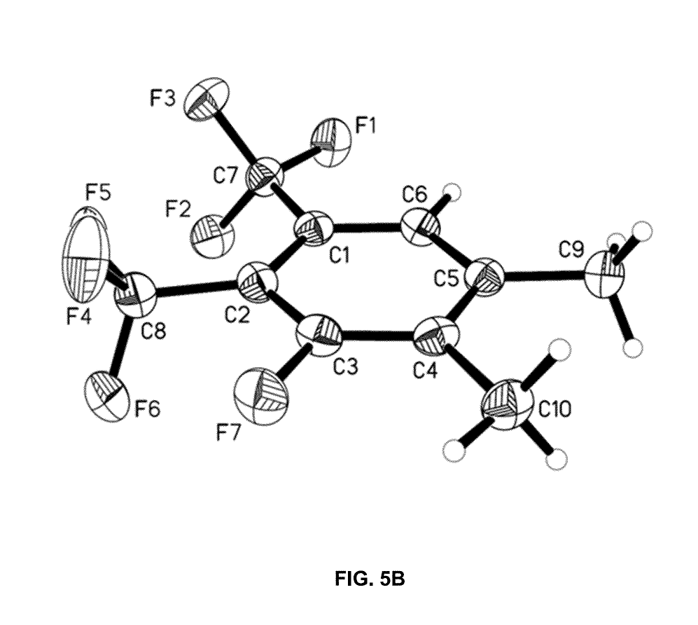

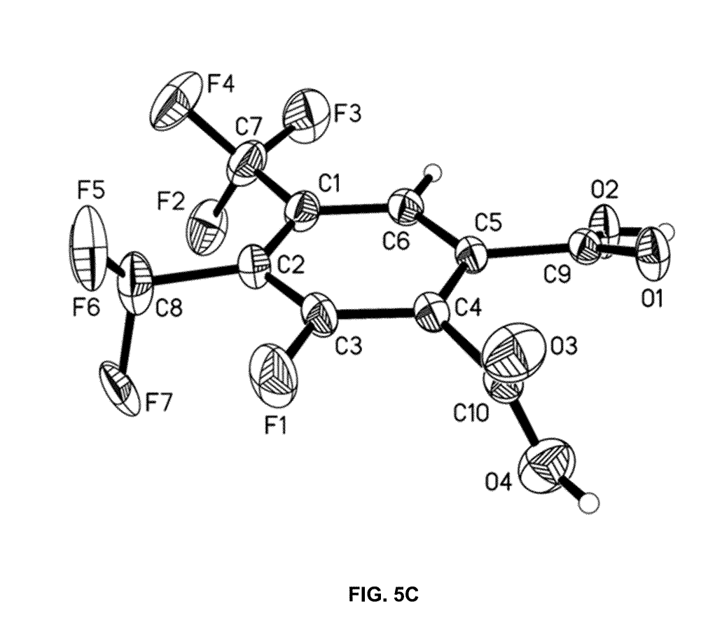

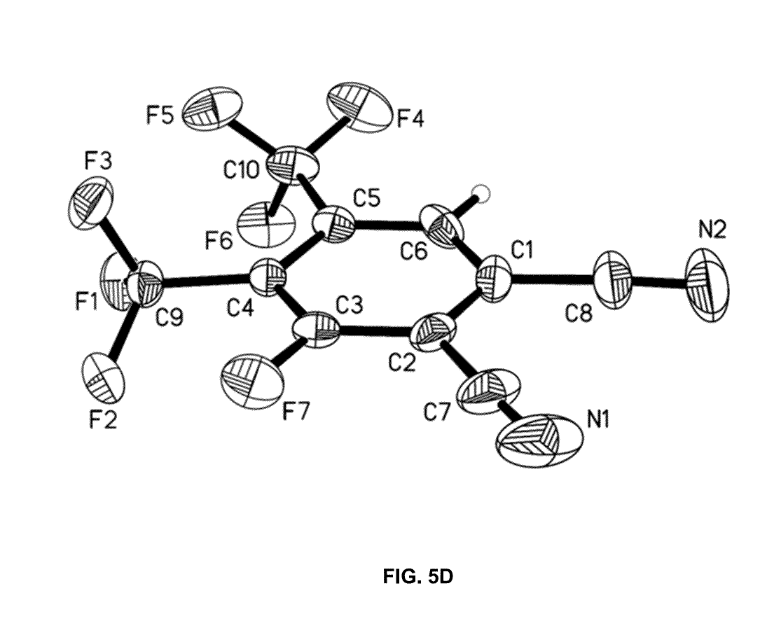

With reference to FIG. 5A, the X-ray structure of exemplary Compound 4 is illustrated with thermal ellipsoids set at about 50% probability.

### Compound 5

With reference to Compound 5 of FIG. 4, an exemplary synthesis and characterization of 1,2-bis(trifluoromethyl)-3-fluoro-4,5-dimethylbenzene (hereinafter “Compound 5”) is depicted. In particular, a solution of Compound 4 (about 2.1 g, 7.7 mmol) in about 25 mL dry DMF is added under stirring at approximately room temperature to a suspension of cesium fluoride (about 3.5 g, 24 mmol) in about 25 mL dry DMF. The mixture is heated under nitrogen to about 120° C. for about 70 h. After cooling, about 80 mL of water is added and the mixture is extracted with diethyl ether (about 3×100 mL). The ether fractions are joined, washed with water (about 3×100 mL), dried over MgSO₄, filtered and then carefully evaporated in vacuo. The crude yellowish oil is purified via flash chromatography on silica gel using hexanes. Evaporation of the first eluted fraction, followed by standing for approximately 2 h at about −20° C. allows for the separation of Compound 5 as colorless crystals in about 34% yield (about 0.67 g). X-ray quality single crystals are obtained by slow evaporation of a refrigerated hexane solution.

Specifically, the exemplary properties of Compound 5 are as follows: Mp: 22-23° C.; ¹H NMR (300 MHz, (CD₃)₂CO): δ 2.33 (3H, s, 5-CH₃), 2.49 (3H, s, 4-CH₃), 7.64 (1H, s, Ph-H); ¹⁹F NMR (282 MHz, (CD₃)₂CO): δ 55.59 (3F, s, 2-CF₃), −57.85 (3F, s, 1-CF₃), −112.18 (1F, m, Ph-F); ¹³C {¹H} NMR (75 MHz, (CD₃)₂CO): δ 11.2 (d, J_{C—F}=6.9 Hz), 20.0 (d, J_{C—F}=2.6 Hz), 113.7 (q, J_{C—F}=34.1 Hz), 123.2 (q, J_{C—F}=273.2 Hz), 123.7 (qd, J_{C—F}=272.6, 3.9 Hz), 125.1 (dq, J_{C—F}=3.0, 6.7 Hz), 125.7 (q, J_{C—F}=31.8 Hz), 131.7 (d, J_{C—F}=17.6 Hz), 146.3 (d, J_{C—F}=6.4 Hz), 159.9 (dq, J_{C—F}=253.6, 2.5 Hz); HRMS (EI): calcd. for [M]⁺ (C₁₀H₇F₇)⁺ 260.0436. found 260.0441.

With reference to FIG. 5B, the X-ray structure of exemplary Compound 5 is illustrated with thermal ellipsoids set at about 50% probability.

### Compound 6

With reference to Compound 6 of FIG. 4, an exemplary synthesis and characterization of 4,5-bis(trifluoromethyl)-3-fluoro-phthalic acid (hereinafter “Compound 6”) is depicted. In particular, Compound 5 (about 1.2 g, 4.6 mmol) is dissolved in about 100 mL acetic acid glacial. To this solution, about 18 mL of sulfuric acid about 96% were added and the mixture is cooled to approximately 15° C. in an ice bath, under stirring. Chromium(VI) trioxide (about 2.1 g, 21 mmol) is added stepwise within approximately 30 min. After the addition, the ice bath is removed and the mixture is allowed to warm to approximately room temperature and then is heated to about 35° C. for about 20 h. Further, the mixture is diluted approximately 1.5 fold with water and about 15 mL methanol is added cautiously in order to destroy the excess CrO₃. The aqueous mixture is extracted with ethyl acetate (about 3×100 mL) and the combined organic fractions are washed with water (about 2×50 mL) and dried over MgSO₄. After filtration, the solvent is evaporated completely under vacuum and the crude yellow product is recrystallized from toluene (about 150 mL), separating Compound 6 as a white crystalline solid in about 53% yield (about 0.78 g).

Specifically, the exemplary properties of Compound 6 are as follows: Mp: 195-196° C.; IR (KBr): 3600-2400, 3031, 2668, 2593, 1729, 1495, 1419, 1281, 1201, 1103, 989, 919, 737 cm⁻¹; ¹H NMR (300 MHz, (CD₃)₂CO): δ 8.36 (1H, s, Ph-H); ¹⁹F NMR (282 MHz, (CD₃)₂CO): δ −56.08 (3F, s, 4-CF₃), −58.44 (3F, s, 5-CF₃), −111.36 (1F, m, Ph-F); ¹³C {¹H} NMR (75 MHz, (CD₃)₂SO): δ 118.4 (qd, J_{C—F}=34.6, 14.2 Hz), 121.0 (q, J_{C—F}=275.9 Hz), 121.6 (qd, J_{C—F}=274.2, 3.5 Hz), 124.7 (octet, J_{C—F}=3.3 Hz), 127.9 (q, J_{C—F}=34.6 Hz), 130.2 (d, J_{C—F}=23.1 Hz), 134.7 (d, T_{C—F}=5.5 Hz), 156.8 (d, J_{C—F}=258.1 Hz), 163.3 (s), 163.8 (s); HRMS (EI): calcd. for [M]⁺ (C₁₀H₃F₇O₄)⁺ 319.9920. found 319.9909.

With reference to FIG. 5C, the X-ray structure for exemplary Compound 6 is illustrated with thermal ellipsoids set at about 50% probability.

### Compound 7

With reference to Compound 7 of FIG. 4, an exemplary synthesis and characterization of 4,5-bis(trifluoromethyl)-3-fluoro-phthalic anhydride (hereinafter “Compound 7”) is depicted. In particular, about 0.58 g (about 1.8 mmol) of Compound 6 are suspended in about 2.5 mL (about 4.1 g, 34.5 mmol) thionyl chloride and heated to approximately 90° C. under stirring for about 3 h. After cooling to approximately room temperature, the excess thionyl chloride is evaporated under an air stream and the product is analyzed and used fresh for phthalimide production. As a result, white Compound 7 is obtained in about 92% yield (about 0.51 g).

Specifically, the exemplary properties of Solid 7 are as follows: Mp: 81-84° C.; IR (KBr): 3037, 1870, 1791, 1623, 1296, 1162, 1100, 910, 732 cm⁻¹; ¹H NMR (300 MHz, (CD₃)₂CO): δ 8.56 (1H, s, Ph-H); ¹⁹F NMR (282 MHz, (CD₃)₂CO): δ −55.68 (3F, s, 4-CF₃), −58.31 (3F, s, 5-CF₃), −107.51 (1F, m, Ph-F). Extreme moisture sensitivity does not allow for well-resolved ¹³C NMR and satisfactory HRMS.

### Compound 8

With reference to Compound 8 of FIG. 4, an exemplary synthesis and characterization of 4,5-bis(trifluoromethyl)-3-fluorophthalimide (hereinafter “Compound 8”) is depicted. In particular, about 0.5 g (about 1.66 mmol) of freshly obtained Compound 7 is mixed intensively with urea (about 0.2 g, 3.32 mmol) and heated under stirring to approximately 140° C. for about 2 h. The white solid product is analyzed and used as received for the next step. As a result, Compound 8 is obtained in about 95% yield (about 0.48 g).

Specifically, the exemplary properties of Compound 8 are as follows: Mp: 184-186° C.; IR (KBr): 3453, 3360, 3251, 3072, 2738, 1744, 1661, 1624, 1329, 1282, 1177, 993, 744, 654 cm⁻¹; ¹H NMR (300 MHz, (CD₃)₂CO): δ 5.39 (1H, br, NH), 8.21 (1H, s, Ph-H); ¹⁹F NMR (282 MHz, (CD₃)₂CO): δ−55.57 (3F, s, 4-CF₃), −58.11 (3F, s, 5-CF₃), −111.33 (1F, m, Ph-F); HRMS (EI): calcd. for [M]⁺ (C₁₀H₂F₇NO₂)⁺ 300.9974. found 300.9975. Low solubility does not allow for a well-resolved ¹³C NMR spectrum.

### Compound 9

With reference to Compound 9 of FIG. 4, an exemplary synthesis and characterization of 4,5-bis(trifluoromethyl)-3-fluorophthalamide (hereinafter “Compound 9”) is depicted. In particular, Compound 8 (about 0.48 g, 1.58 mmol) is powdered, suspended in about 20 mL ammonium hydroxide about 28% and stirred for approximately 18 h. The mixture becomes a thick paste, which is filtered off and dried under vacuum to afford white Compound 9 in about 70% yield (about 0.35 g).

Specifically, the exemplary properties of Compound 9 are as follows: Mp: 203-204° C.; IR (KBr): 3461, 3422, 3305, 3025, 1713, 1610, 1356, 1128, 766 cm⁻¹; ¹H NMR (300 MHz, (CD₃)₂CO): δ 7.27 (1H, s, 1-CONH₂), 7.48 (1H, s, 2-CONH₂), 7.62 (1H, s, 1-CONH₂), 7.74 (1H, s, 2-CONH₂), 8.06 (1H, s, Ph-H); ¹⁹F NMR (282 MHz, (CD₃)₂CO): δ −55.59 (3F, s, 4-CF₃), −58.26 (3F, s, 5-CF₃), 110.59 (1F, m, Ph-F); HRMS (EI): calcd. for [M]⁺ (C₁₀H₅F₇N₂O₂)⁺318.0239. found 318.0232.

### Compound 10

With reference to Compound 10 of FIG. 4, an exemplary synthesis and characterization of 4,5-bis(trifluoromethyl)-3-fluorophthalonitrile (hereinafter “Compound 10”) is depicted. In particular, Compound 9 (about 0.1 g, 0.31 mmol) is dissolved in about 2 mL dry DMF. The solution is cooled to approximately −10° C. and a solution of about 72 μL (about 0.12 g, 1 mmol) thionyl chloride in about 2 mL dry DMF is dropped within approximately 15 min. After stirring for about 30 min at approximately −10° C., the mixture is allowed to warm to about room temperature and stirred overnight. The brownish solution is then given to about 50 g ice and stirred for approximately 15 min. The crude solid is filtered off, dried under air, re-dissolved in acetone and filtered again from brown impurities. Evaporation of the acetone solution gives a gray Compound 10 in about 52% yield (about 0.045 g).

Specifically, the exemplary properties of Compound 10 are as follows: Mp: 35-36° C.; IR (KBr): 3128, 3078, 2247, 1739, 1621, 1573, 1430, 1343, 1183, 1120, 1014, 930, 684 cm⁻¹; ¹H NMR (300 MHz, (CD₃)₂CO): δ 8.71 (1H, s, Ph-H); ¹⁹F NMR (282 MHz, (CD₃)₂CO): δ −56.29 (3F, s, 4-CF₃), −58.69 (3F, s, 5-CF₃), −99.35 (1F, m, Ph-F); ¹³C {¹H} NMR (75 MHz, (CD₃)₂CO): δ 110.5 (s), −112.7 (d, J_{C—F}=20.6 Hz), 113.9 (d, J_{C—F}=2.3 Hz), 121.4 (q, J_{C—F}=275.9 Hz), 121.8 (d, J_{C—F}=12.3 Hz), 122.0 (qd, J_{C—F}=274.9, 3.3 Hz), 122.3 (s), 129.9 (dq, J_{C—F}=4.5, 6.8 Hz), 134.3 (q, J_{C—F}=34.8 Hz), 162.6 (dq, J_{C—F}=270.0, 2.1 Hz); HRMS (EI): calcd. for [M]⁺ (C₁₀HF₇N₂)⁺ 282.0028. found 282.0037.

With reference to FIG. 5D, the X-ray structure of exemplary Compound 10 is illustrated with thermal ellipsoids set at about 50% probability.

### Compound 11

With reference to Compound 11 of FIG. 4, an exemplary synthesis and characterization of 2,3,9,10,16,17,23,24-octakis-(trifluoromethyl)-tetrafluorophthalocyaninato-zinc(II) (hereinafter “Compound 11”) is depicted. In particular, about 0.24 g (about 0.85 mmol) of Compound 10, about 0.08 g (about 0.43 mmol) zinc(II) acetate dihydrate and about 2 mL nitrobenzene are mixed in an approximately 10 mL glass reaction vessel, sealed with a Teflon cap and heated under microwave radiation for about 15 min at approximately 200° C. After cooling down, the blue-green solid is dissolved in ethyl acetate and purified via flash chromatography on silica gel (mesh size approximately 35-70) using first ethyl acetate and then acetonehexane (about 1:1) as eluents. Evaporation of the solvent affords dark blue Compound 11 in about 38% yield (about 0.096 g). X-ray quality single crystals are then obtained by slow evaporation of an acetoneacetonitrile (about 1:1) solution.

Specifically, the exemplary properties of Compound 11 are as follows: Mp>300° C.; TGA: sublimes at 475° C.; UV-Vis (CHCl₃): λₘₐₓ (log ε) 675 (5.25), 647 (4.42), 609 (4.44), 378 (4.56) nm (L mol⁻¹ cm⁻¹); IR (KBr): 2928, 1633, 1414, 1285, 1161, 942, 720 cm⁻¹; ¹H NMR (300 MHz, (CD₃)₂CO): δ 9.11-9.46 (4H, m, Ph-H); ¹⁹F NMR (282 MHz, (CD₃)₂CO): δ −53.48 (12F, br, CF₃), −56.72 (12F, br, CF₃), −109.09 (4F, br, Ph-F); HRMS (APCI+): calcd. for [M+H]⁺ (C₄₀H₅F₂₈N₈Zn)_{±}1192.9476. found 1192.9491.

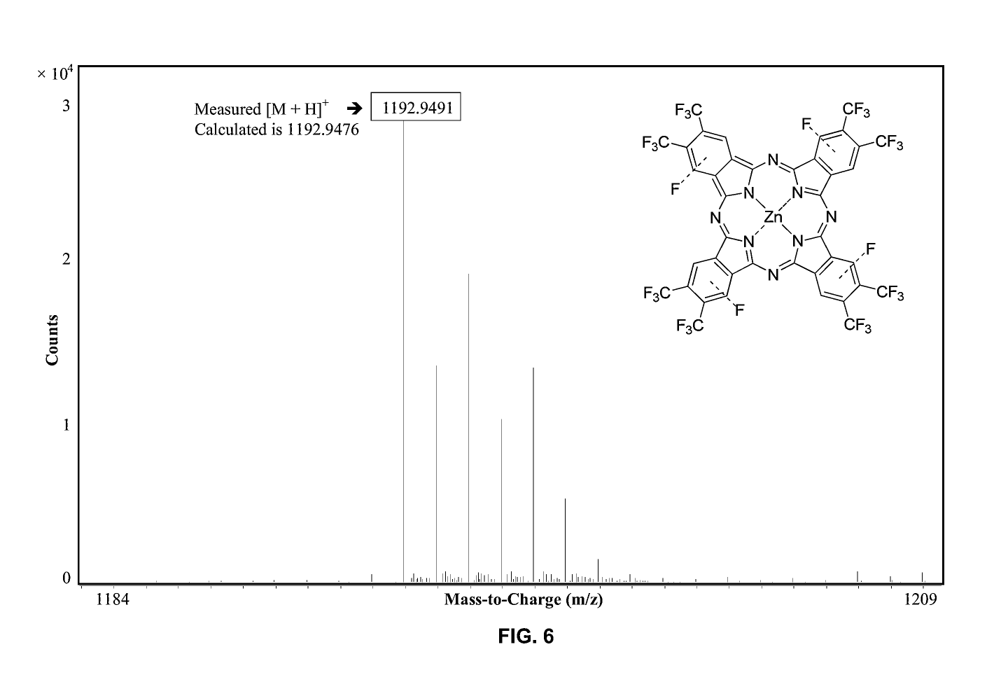

With reference to FIG. 6, the measured exact mass spectrum (positive ion APCI) and isotope pattern of [M+H]⁺ for F₂₈H₄PcZn are depicted, indicating the calculated value for [M+H]⁺.

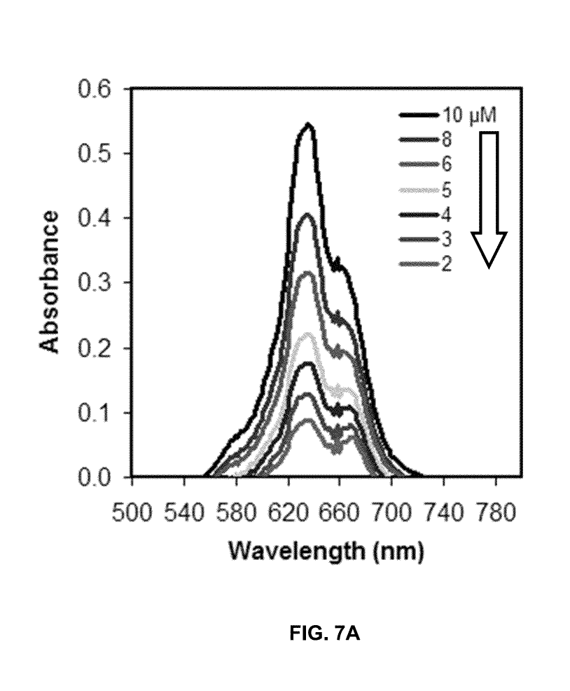

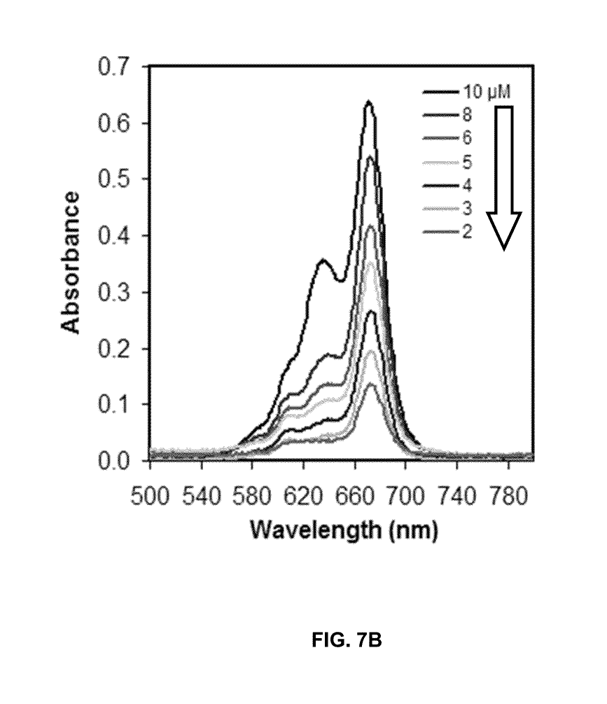

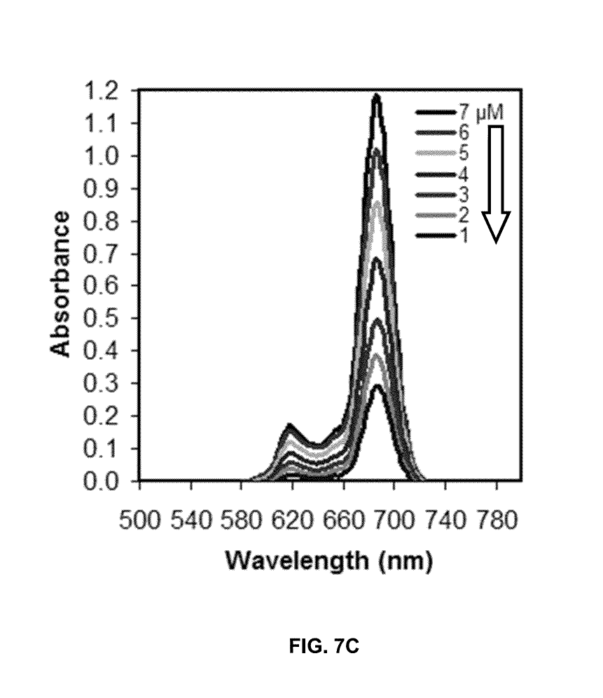

Turning now to FIGS. 7A-C, UV-Vis data comparison is displayed of partially aggregated F₂₈H₄PcZn with sterically non-hindered F₁₆PcZn and sterically hindered F₆₄PcZn. It should be noted that the spectra of FIGS. 7A-C have been recorded in acetone. Further, the UV-Vis data for F₂₈H₄PcZn is displayed in FIG. 7A, for F₁₆PcZn in FIG. 7B, and for F₆₄PcZn in FIG. 7C.

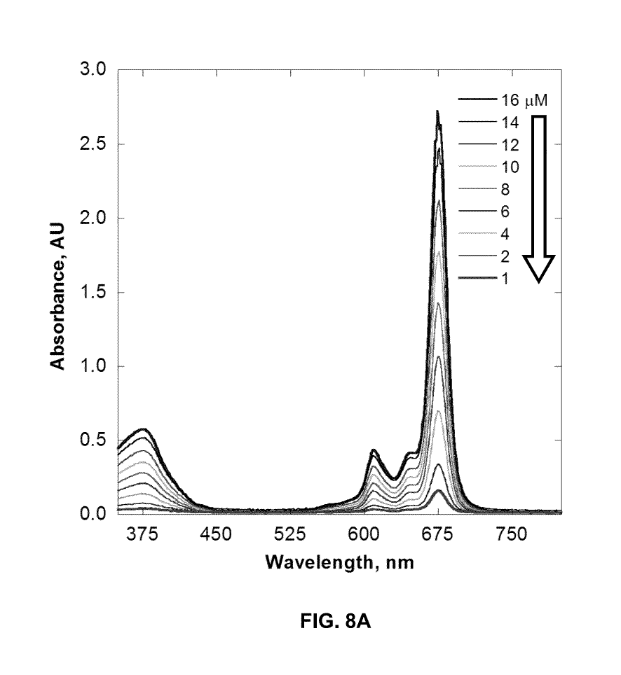

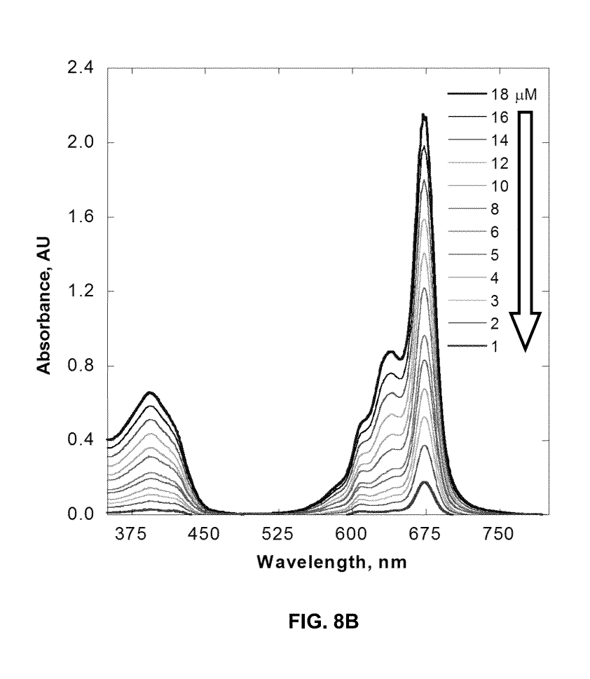

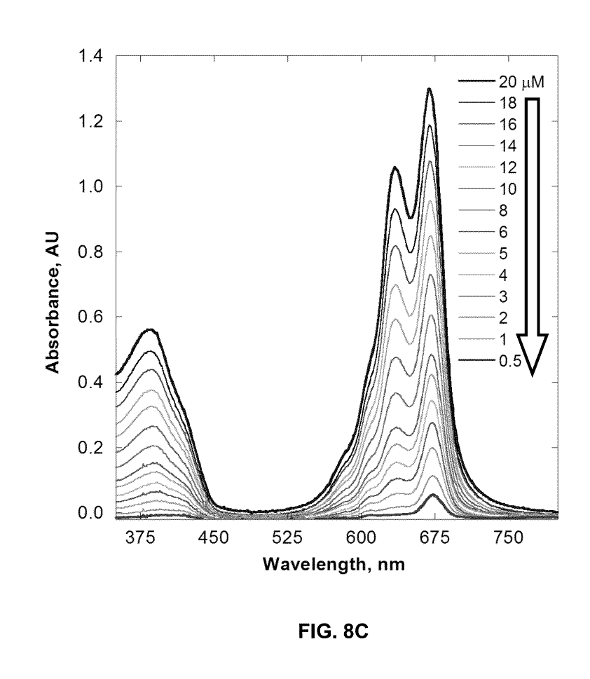

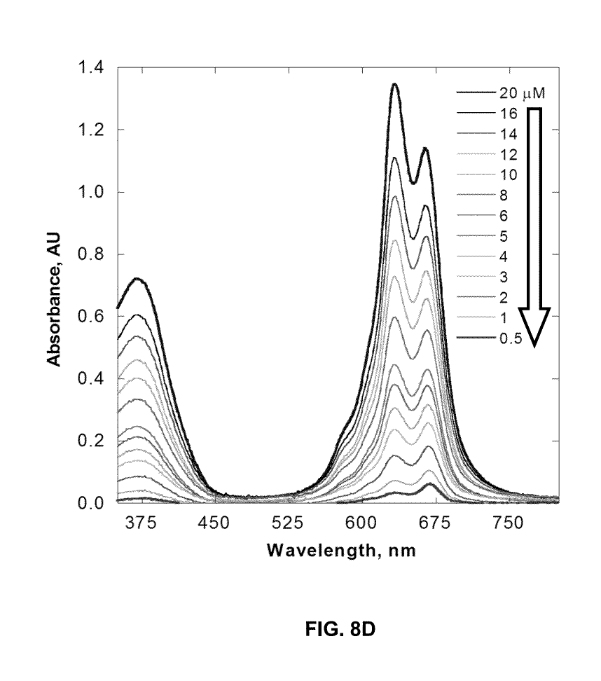

With reference to FIGS. 8A-D, UV-Vis electronic absorption spectra of F₂₈H₄PcZn are shown, depicting strong solvent-dependent aggregation. In particular, FIG. 8A illustrates a spectrum recorded in chloroform, in which F₂₈H₄PcZn is a monomer with minimal aggregation, FIG. 8B illustrates a spectrum recorded in ethyl acetate, in which F₂₈H₄PcZn is mostly a monomer, FIG. 8C illustrates a spectrum recorded in acetone, in which F₂₈H₄PcZn displays intermediate aggregation, and FIG. 8D illustrates a spectrum recorded in ethanol, in which F₂₈H₄PcZn is mostly aggregated.

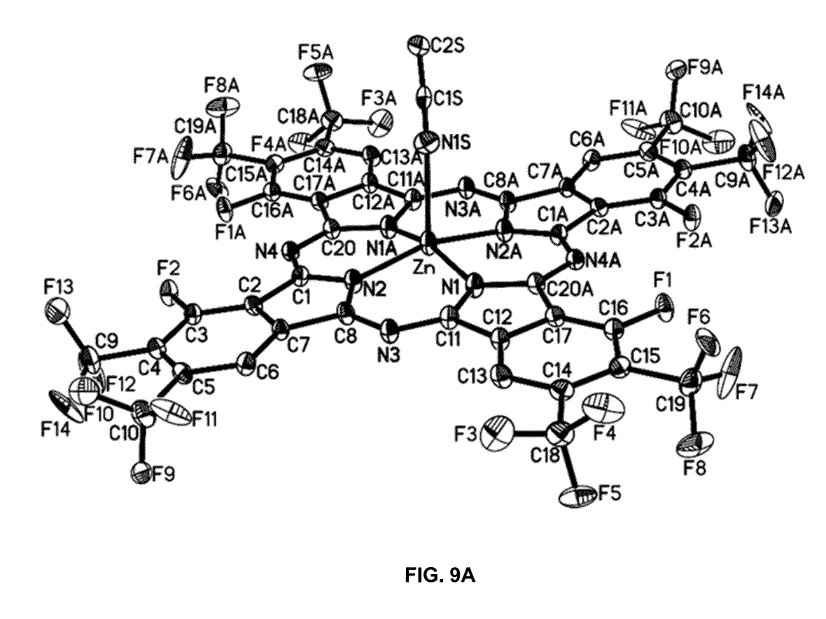

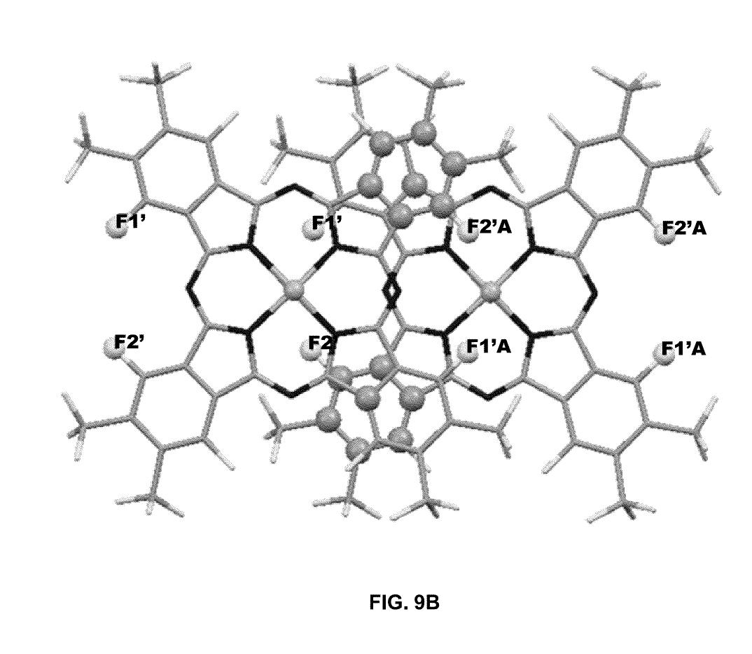

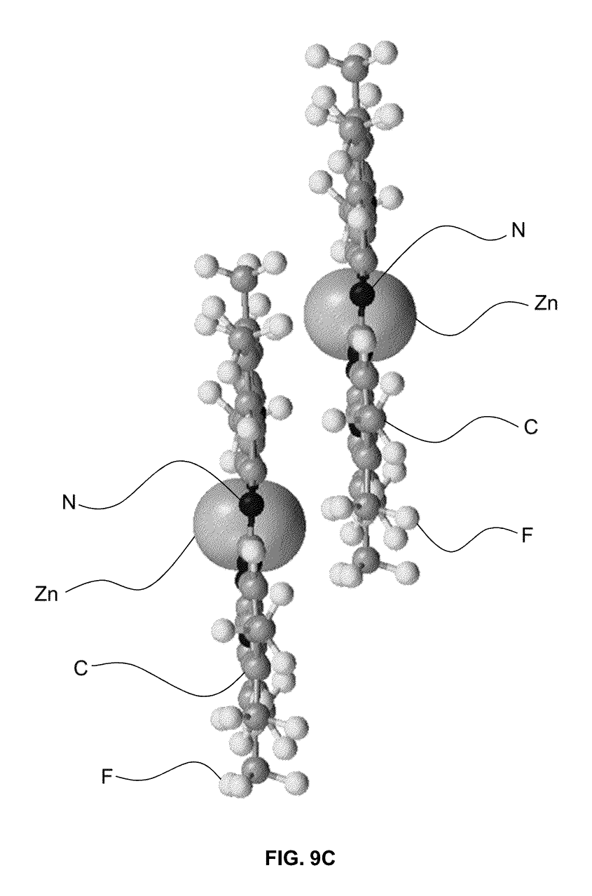

Turning now to FIG. 9A, X-ray structures of another exemplary embodiment of F₂₈H₄PcZn(CH₃CN) are depicted showing metal-coordinated acetonitrile with H atoms omitted for clarity. The thermal ellipsoids are depicted at about 40% probability. It should be noted that the presence of the aromatic F at both non-peripheral positions in a non-equivalent ratio indicates the presence of at least two stereoisomers. While a statistical disorder about the ring plane may be less likely, it is not impossible. For clarity, FIG. 9A only illustrates the major population of F atoms on each ring. With reference to FIG. 9B, it illustrates the top view of the π-π stacking region of two adjacent molecules of exemplary F₂₈H₄PcZn, with the darker atoms belonging to the upper molecule. FIG. 9C further illustrates a ball-and-stick representation of the side view of the aggregation of exemplary F₂₈H₄PcZn in a solid state.

### Compound 12

With reference to Compound 12 of FIG. 4, an exemplary synthesis and characterization of 2,3,9,10,16,17,23,24-octakis-(trifluoromethyl)-tetrafluorophthalocyaninato-cobalt(II) (hereinafter “Compound 12”) is depicted. In particular, Compound 12 is prepared and purified in a similar manner to Compound 11, using about 0.035 g (about 0.12 mmol) of Compound 10, about 0.012 g (about 0.07 mmol) cobalt(II) acetate tetrahydrate and about 2 mL nitrobenzene. The brute blue-green solid obtained after evaporation of the ethyl acetate fraction is treated with about 50 mL acetone, filtered and dried under air to afford purple-violet Compound 12 in about 51% yield (about 0.018 g).

Specifically, the exemplary properties of Compound 12 are as follows: Mp>300° C.; UV-Vis (CHCl₃): λₘₐₓ (log ε) 665 (4.56), 602 (3.92), 347 (4.24) nm (L mol⁻¹ cm⁻¹); ¹⁹F NMR (282 MHz, (CD₃)₂CO): δ −53.87 (12F, br, CF₃), −56.72 (12F, br, CF₃), −108.94 (4F, br, Ph-F); HRMS (APCI+): calcd. for [M+H]⁺ (C₄₀H₅F₂₈N₈Co)⁺ 1187.9517. found 1187.9564.

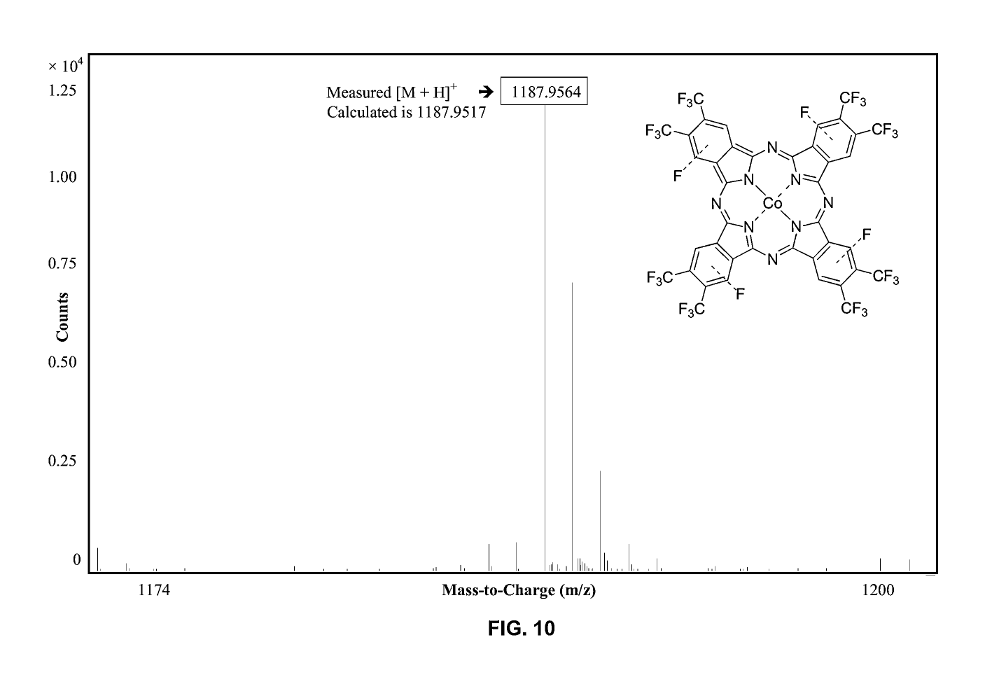

With reference to FIG. 10, the measured exact mass spectrum (positive ion APCI) and isotope pattern for [M+H]⁺ for F₂₈H₄PcCo are depicted, indicating the calculated value for [M+H]⁺.

### Example 2

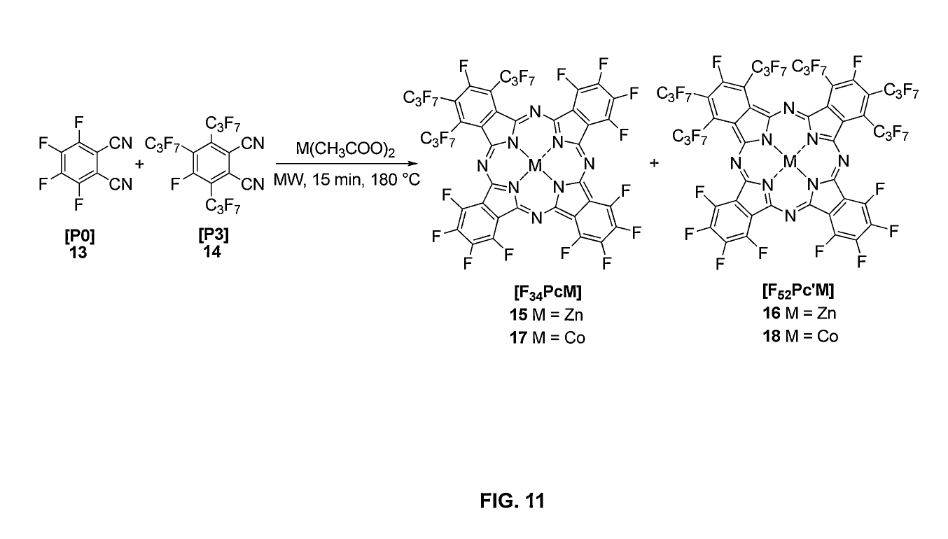

With reference to FIG. 11, an exemplary synthesis scheme for production of asymmetric F₃₄PcM and F₅₂Pc′M is depicted, showing the results of the combination of precursors P0 and P3 and including Compounds 15, 16, 17 and 18, which will be discussed in greater detail below. It should be noted that the F₃₄PcM and F₅₂Pc′M compounds are obtained together. Further, the approximately 3:1 molecular tetramerization of the two precursors yields F₃₄PcM compound, while the approximately 2:2 tetramerization yields F₅₂Pc′M compound. As should be noted, the Pc′ notation is used to differentiate two F₅₂Pc compositionals (see, e.g., FIGS. 3A-E). In one experimental embodiment of this class of molecules the metal used is Zn. As will be apparent to one of ordinary skill in the art, the present embodiment embraces the use of multiple other metals as the synthesis scheme is not metal specific and would include, but not be limited to, other metals with ionic radii that would be coordinated by the four nitrogen atoms of the phthalocyanines, i.e., Co, Fe, Mg, Cu, and the like.

### Compounds 15 and 16

With reference to Compounds 15 and 16, an exemplary synthesis and characterization of F₃₄PcZn (hereinafter “Compound 15”) and F₅₂Pc′Zn (hereinafter “Compound 16”) are depicted. In particular, twenty (20) thick walled glass reaction vessels (about 10 mL volume) are charged each with about 0.4 g (about 0.62 mmol) perfluoro-3,5,6-triisopropyl phthalonitrile, (depicted in FIG. 11 as P3 and hereinafter “Compound 14”), about 0.04 g (about 0.2 mmol) tetrafluorophthalonitrile (depicted in FIG. 11 as P0 and hereinafter “Compound 13”) and about 0.04 g (about 0.22 mmol) zinc(II) acetate dihydrate. Then, catalytic amounts of ammonium molibdate, and about 1 mL nitrobenzene are added to each vial. The sealed vessels are heated in a microwave reactor at approximately 180° C. for about 15 min. The crude solid of each vial is extracted with about 50 mL ethyl acetate, the organic fractions are combined, concentrated in vacuo and adsorbed to silica gel (mesh size approximately 70-230). Gel filtration using an acetonehexane approximately 2:98 mixture (v/v) allows for the complete separation of nitrobenzene, unreacted Compound 14 and most yellowish impurities. The resulting blue-green solid is collected and subjected to column chromatography under gradually increasing solvent polarity. The rest of yellow impurities are removed with acetonehexane approximately 2:98 mixture, followed by the separation of the green exemplary F₅₂Pc′Zn, eluted with an approximately 10:90 mixture, the royal blue exemplary F₃₄PcZn at approximately 20:80 polarity, and finally the dark blue exemplary F₁₆PcZn as a side product using an approximately 40:60 mixture (v/v). The three colored fractions are evaporated and re-purified by gel filtration on short columns, eluting with the corresponding mixtures used for their initial separation. Removal of the solvent and drying of the compounds allows for isolation of exemplary F₅₂Pc′Zn in about 13% yield (about 0.42 g), exemplary F₃₄PcZn in about 16% yield (about 0.26 g) and exemplary F₁₆PcZn in about 14% yield (about 0.1 g), all based on starting material Compound 13.

Specifically, the exemplary properties for Compound 15, i.e., F₃₄PcZn, are as follows: Mp>300° C.; UV-vis (CHCl₃): λₘₐₓ (log ε) 689 (5.09), 672 (4.99), 632 (4.44), 614 (4.41), 365 (4.69) nm (L mol⁻¹ cm⁻¹); IR (KBr): 1522, 1489, 1383, 1282, 1236, 1133, 964 cm⁻¹; ¹⁹F NMR (282 MHz, (CD₃)₂CO): δ −69.05 (6F, br, CF₃), −72.25 (12F, s, CF₃), −97.12 (1F, s, Ar—F), −131.4 (1F, s, CF), −135.09 (1F, d, Ar—F), −139.18 to −141.66 (5F, m, Ar—F), −149.92 to −151.6 (6F, m, Ar—F), −161.39 (1F, d, CF), −165.99 to +170.18 (1F, m, CF); HRMS (APCI+): calcd. for [M+H]⁺ (C₄₁HF₃₄N₈Zn)⁺ 1314.9067. found 1314.9080.

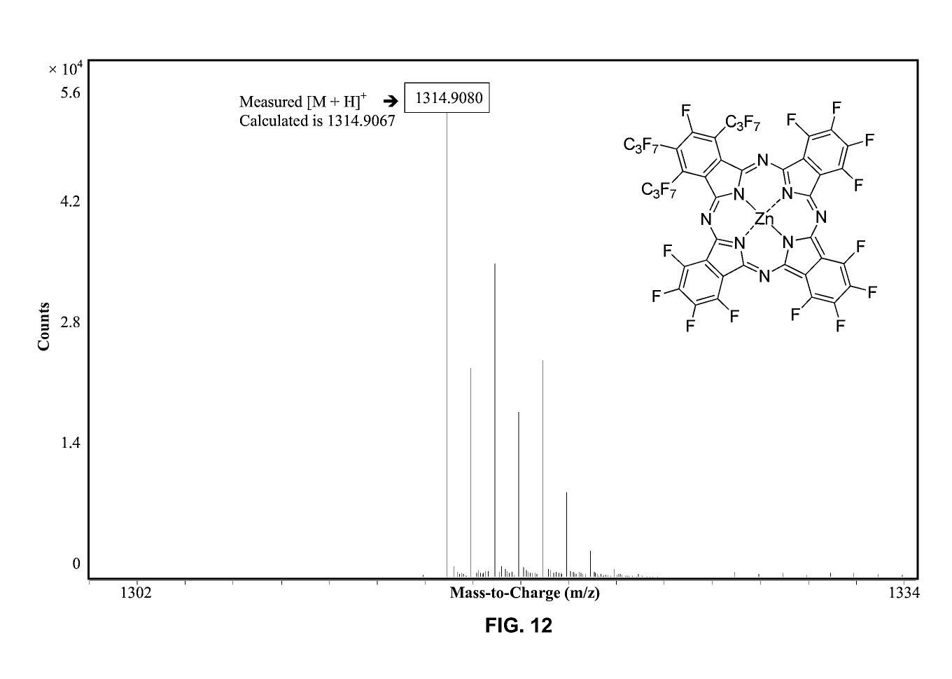

With reference to FIG. 12, the measured exact mass spectrum (positive ion APCI) and isotope pattern of [M+H]⁺ for F₃₄PcZn are depicted, indicating the calculated value for [M+H]⁺.

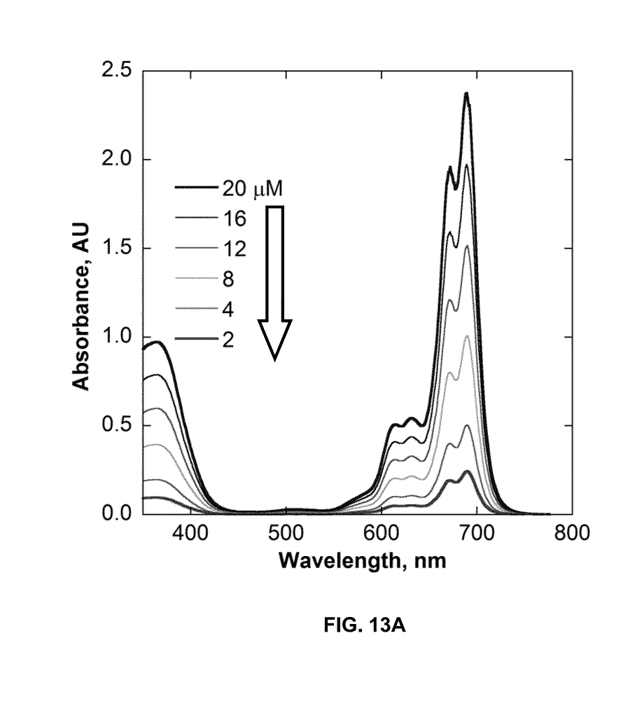

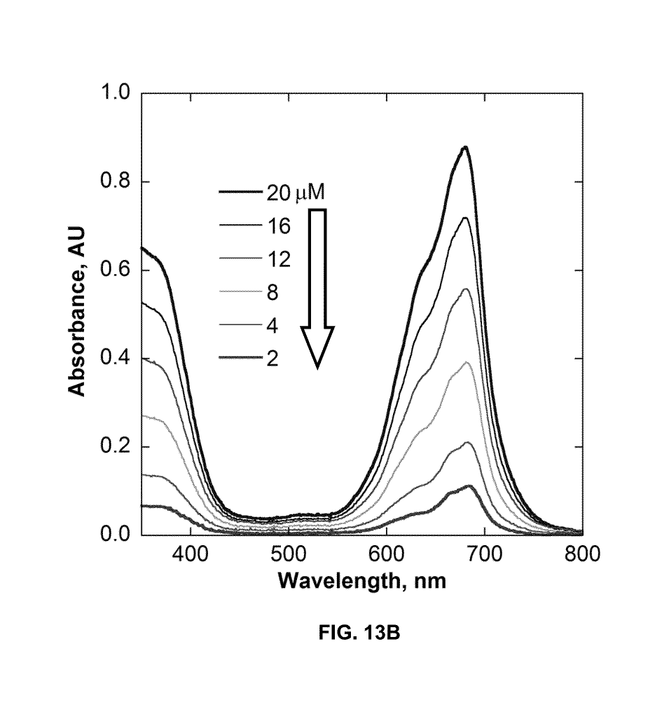

Turning now to FIGS. 13A and B, the UV-Vis electronic absorption spectra of F₃₄PcZn are illustrated, showing solvent-dependent aggregation. In particular, FIG. 13A illustrates a spectrum recorded in chloroform, in which F₃₄PcZn is a monomer, and FIG. 13B illustrates a spectrum recorded in ethanol, in which F₃₄PcZn displays a significant degree of dimerization.

Further, the exemplary properties for Compound 16, i.e., F₅₂Pc′Zn, are as follows: Mp>300° C.; UV-vis (CHCl₃): λₘₐₓ (log ε) 701 (5.10), 674 (4.97), 640 (4.62), 615 (4.44), 372 (4.78) nm (L mol⁻¹ cm⁻¹); IR (KBr): 1523, 1489, 1375, 1287, 1236, 1166, 1127, 1050, 966, 939, 737 cm⁻¹; ¹⁹F NMR (282 MHz, (CD₃)₂CO): δ −63.23 (3F, br, CF₃), −68.52 (3F, br, CF₃), −70.69 to −76.31 (30F, m, CF₃), −97.56 (2F, br, Ar—F), −130.85 (1F, d, CF), −137.91 to −141.55 (5F, m, Ar—F), −151.23 to −152.76 (4F, m, Ar—F), −161.49 (1F, d, CF), −166.47 to −170.15 (3F, m, CF); HRMS (APCI+): calcd. for [M+H]⁺ (C₅₀HF₅₂N₈Zn)⁺ 1764.8780. found 1764.8804.

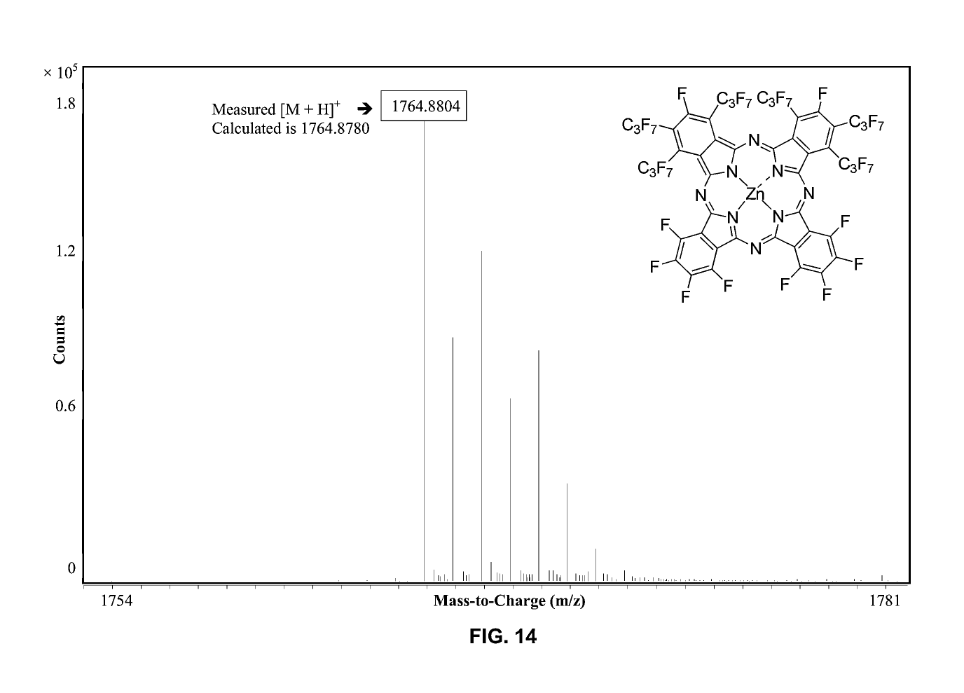

With reference to FIG. 14, the measured exact mass spectrum (positive ion APCI) and isotope pattern of [M+H]⁺ for F₅₂Pc′Zn are depicted, indicating the calculated value for [M+H]⁺.

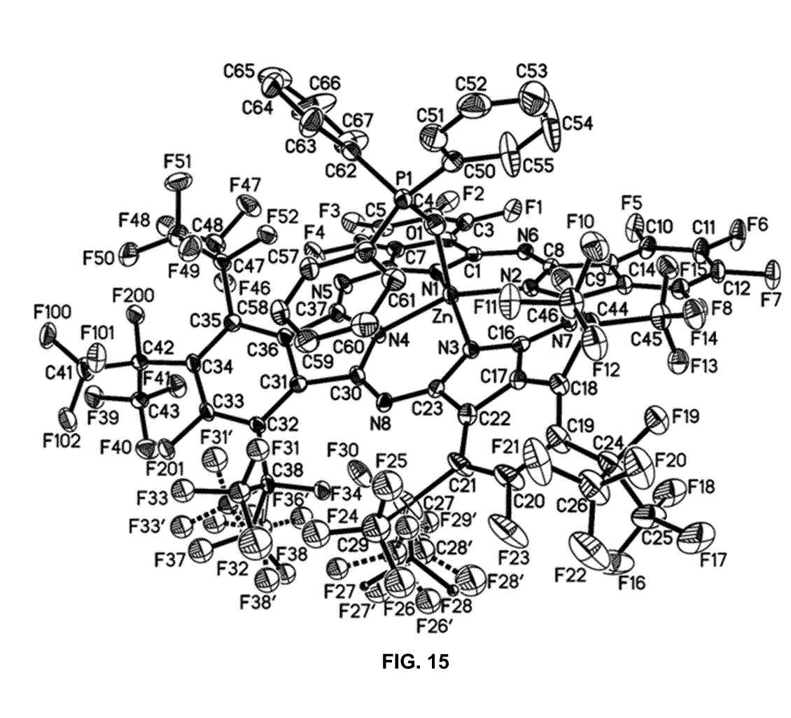

Turning now to FIG. 15, the X-ray structure of F₅₂Pc′Zn(OPPh₃) is depicted, showing a metal-coordinated triphenyl phosphine oxide molecule. The thermal ellipsoids are plotted at about 35% probability and rotational disorder of the CF₃ groups of i-C₃F₇ is present, specifically shown as dashed lines.

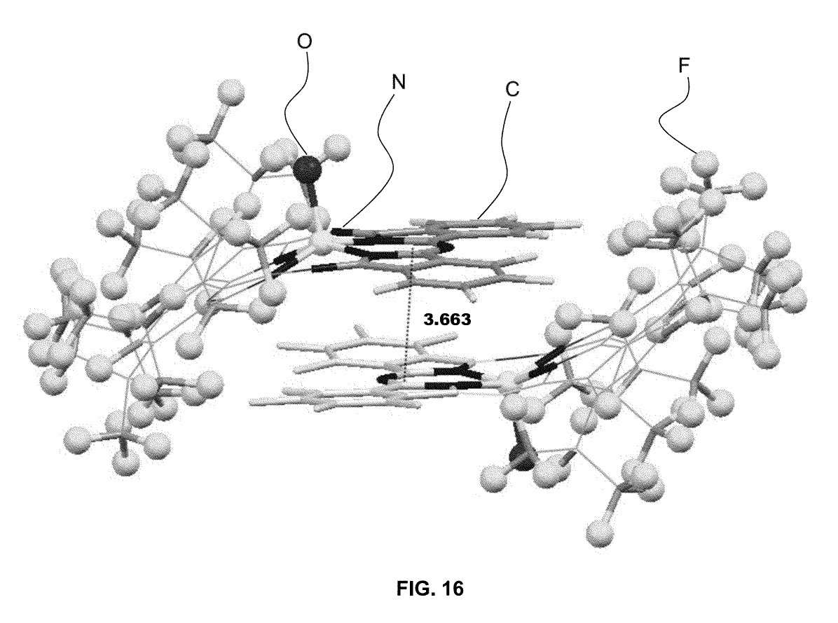

With reference to FIG. 16, the side view of the aggregation in solid state of F₅₂Pc′Zn is illustrated. In particular, the toluene molecules in the crystalline lattice and the atoms of coordinated triphenyl phosphine oxide, except oxygen, have been omitted. Further, the i-C₃F₇ groups are shown in ball-and-stick representation and the interplanar stacking distance, approximately 3.663 Å, proves the existence of π-π interactions.

### Compounds 17 and 18

With reference to Compounds 17 and 18, an exemplary synthesis and characterization of F₃₄PcCo (hereinafter “Compound 17”) and F₅₂Pc′Co (hereinafter “Compound 18”) is depicted. In particular, Compounds 17 and 18 are prepared similarly to Compounds 15 and 16, using sixteen (16) glass vessels, each charged with about 0.3 g (about 0.47 mmol) of Compound 14, about 0.05 g (about 0.25 mmol) of Compound 13 and about 0.045 g (about 0.18 mmol) cobalt(II) acetate tetrahydrate. Microwave heating is performed for approximately 12 min at about 185° C. Initial purification of the brute solid by gel filtration is done with a toluenehexane approximately 1:9 mixture (v/v). The rest of the separations are carried out as described for Compounds 15 and 16. Evaporation of the eluted fractions and drying to constant weight allows for isolation of green exemplary F₅₂Pc′Co (Compound 18) in about 1.5% yield (about 0.05 g), exemplary F₃₄PcCo (Compound 17) in about 11% yield (about 0.19 g) and exemplary F₁₆PcCo as a side product in about 10% yield (about 0.084 g), based on starting material Compound 13. About 4.5 g of Compound 14 are recovered following the initial separation (about 90% of initial amount). X-ray quality single crystals for exemplary F₃₄PcCo are obtained by slow evaporation of an acetonitriletoluene approximately 1:1 solution.

Specifically, the exemplary properties of Compound 17, i.e., F₃₄PcCo, are as follows: Mp>300° C.; UV-vis (CHCl₃): λₘₐₓ (log ε) 680 (4.52), 667 (4.50), 611 (4.03) nm (L mol⁻¹ cm⁻¹); ¹⁹F NMR (282 MHz, (CD₃)₂CO): δ −63.58 (3F, br, CF₃), −67.36 (3F, s, CF₃), −68.75 to −76.79 (12F, m, CF₃), −100.98 (1F, br, Ar—F), −132.36 (1F, s, CF), −137.64 (1F, d, Ar—F), −139.44 to −142.63 (5F, m, Ar—F), −155.92 to −157.62 (6F, m, Ar—F), −165.55 (1F, d, CF), −169.46 (1F, br, CF); HRMS (APCI−): calcd. for [M]⁻ (C₄₁F₃₄N₈Co)⁻ 1308.9040. found 1308.9032.

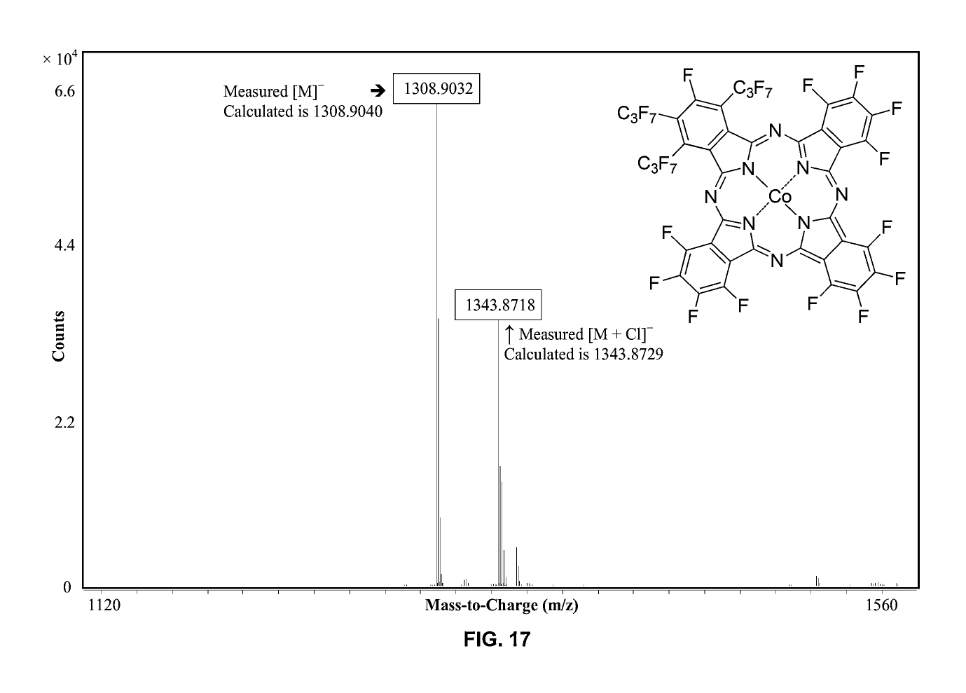

With reference to FIG. 17, the measured exact mass spectrum (negative ion APCI) and isotope pattern of [M]⁻ for F₃₄PcCo are depicted, indicating the calculated value for [M]⁻.

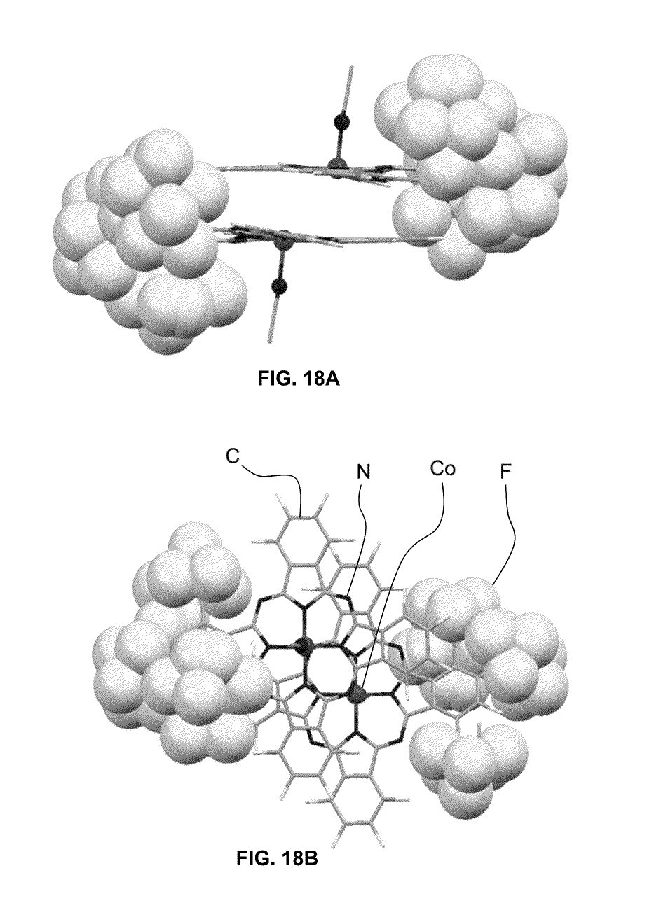

Turning now to FIG. 18A, the side view of the aggregation in solid state of F₃₄PcCo is illustrated. In particular, the toluene molecules in the crystalline lattice and the H atoms of coordinated acetonitrile have been omitted and the i-C₃F₇ groups are depicted as van der Waals spheres. The interplanar stacking distance, approximately 3.25 Å, illustrates the existence of π-π interactions. With reference to FIG. 18B, a top view of the π-π stacking region of two adjacent molecules of F₃₄PcCo is depicted.

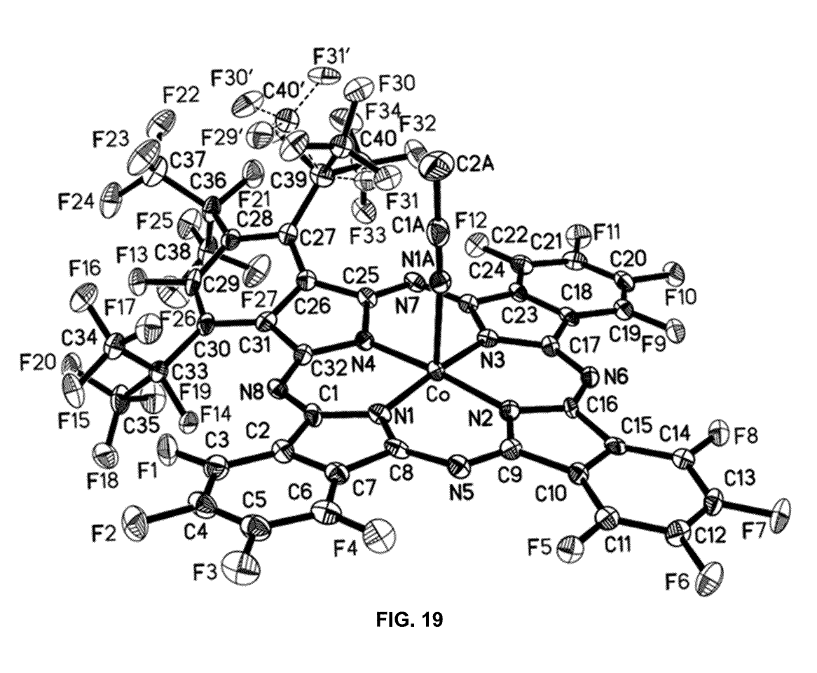

With reference to FIG. 19, the X-ray structure of F₃₄PcCo(CH₃CN) is depicted, showing a metal-coordinated acetonitrile molecule. In particular, the thermal ellipsoids are plotted at about 40% probability and rotational disorder of the CF₃ groups of i-C₃F₇ is present, as is shown by the dashed lines.

Further, the exemplary properties of Compound 18, i.e., F₅₂Pc′Co, are as follows: Mp>300° C.; UV-vis (CHCl₃): λₘₐₓ (log ε) 686 (4.62), 615 (4.18), 334 (4.58) nm (L mol⁻¹ cm⁻¹); ¹⁹F NMR (282 MHz, (CD₃)₂CO): δ −63.62 (3F, br, CF₃), −67.01 to −76.28 (33F, m, CF₃), −90.0 to −110.0 (2F, br, Ar—F), −137.5 to −147.5 (6F, m, Ar—F), −155.0 to −159.5 (4F, br, Ar—F), −165.82 (1F, m, CF), −169.76 to −171.73 (3F, m, CF); HRMS (APCI−): calcd. for [M]⁻ (C₅₀F₅₂N₈Co)⁻ 1758.8753. found 1758.8763.

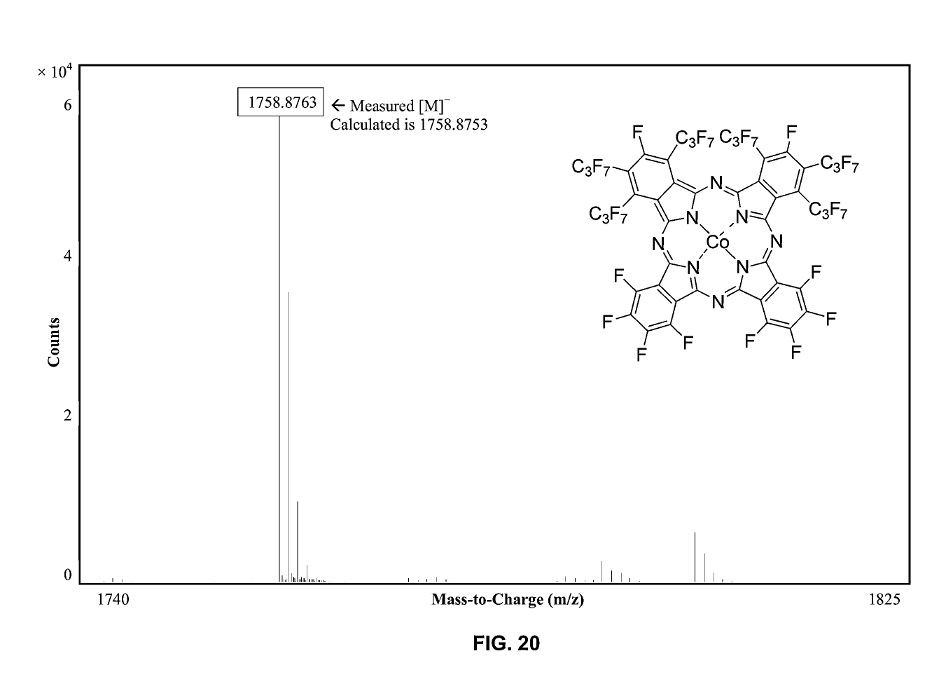

With reference to FIG. 20, the measured exact mass spectrum (negative ion APCI) and isotope pattern of [M]⁺ for F₅₂Pc′Co are depicted, indicating the calculated value for [M]⁻.

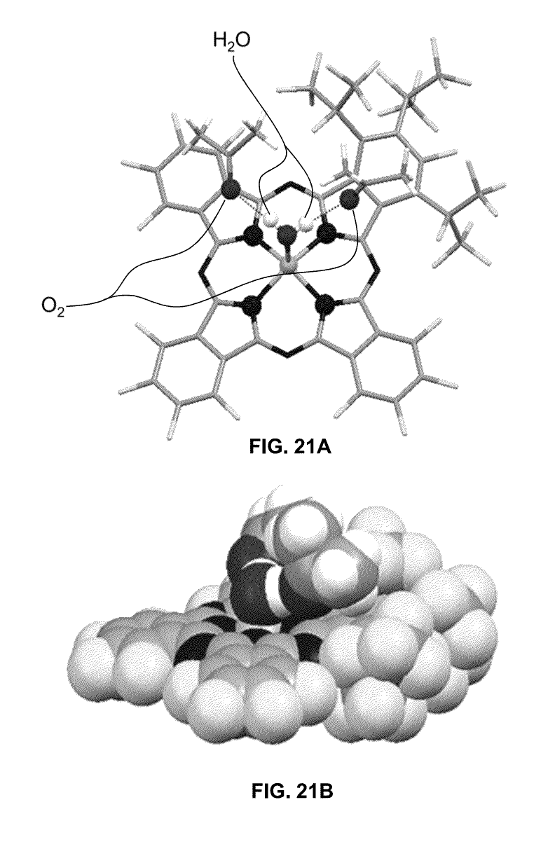

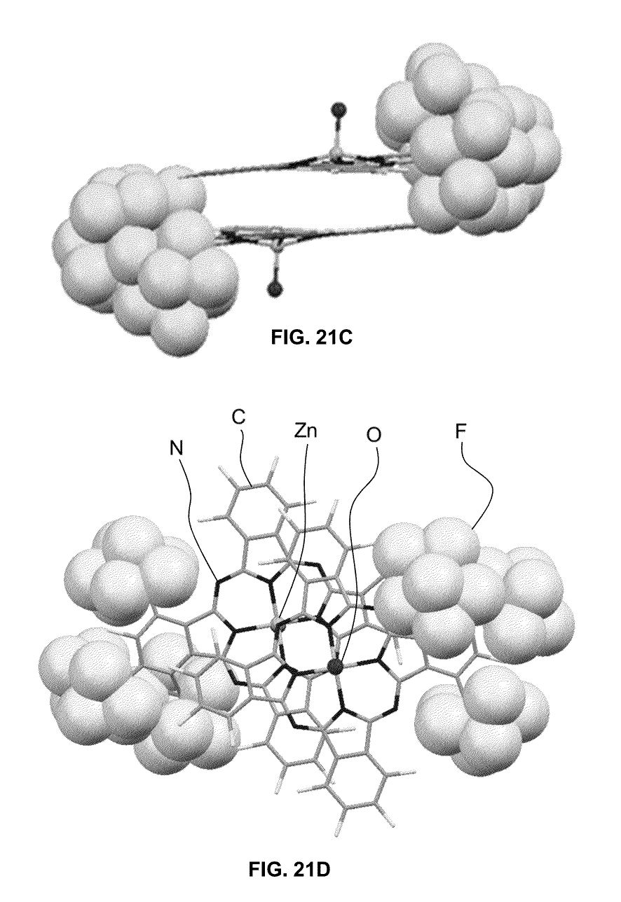

Turning now to FIG. 21A, a ball-and-stick representation of F₃₄PcZn(H₂O).((CH₃)₂CO)₂ is depicted showing H-bonding between the H atoms of H₂O and the oxygen atoms (O₂) of the two acetone molecules. FIG. 21B is a van der Waals representation of the exemplary F₃₄PcZn(H₂O).((CH₃)₂CO)₂. FIG. 21C illustrates the side view of the aggregation in solid state of exemplary F₃₄PcZn. The acetone molecules in the crystalline lattice and the H atoms of coordinated H₂O have been omitted for clarity. The i-C₃F₇ groups are depicted as van der Waals spheres. The interplanar stacking distance, about 3.393 Å, demonstrates the existence of π-π interactions. Further, FIG. 21D illustrates a top view of the π-π stacking region of two adjacent molecules of exemplary F₃₄PcZn.

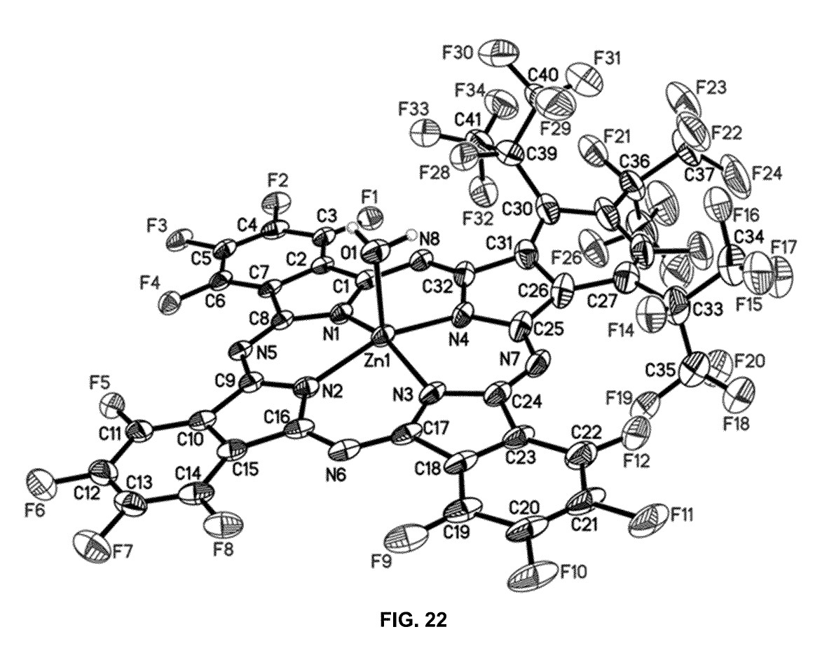

Turning now to FIG. 22, the X-ray structure of F₃₄PcZn(H₂O) is illustrated, showing a metal-coordinated water molecule. In particular, the acetone molecules in the crystalline lattice have been omitted. The thermal ellipsoids of FIG. 22 are plotted at about 40% probability.

### Example 3

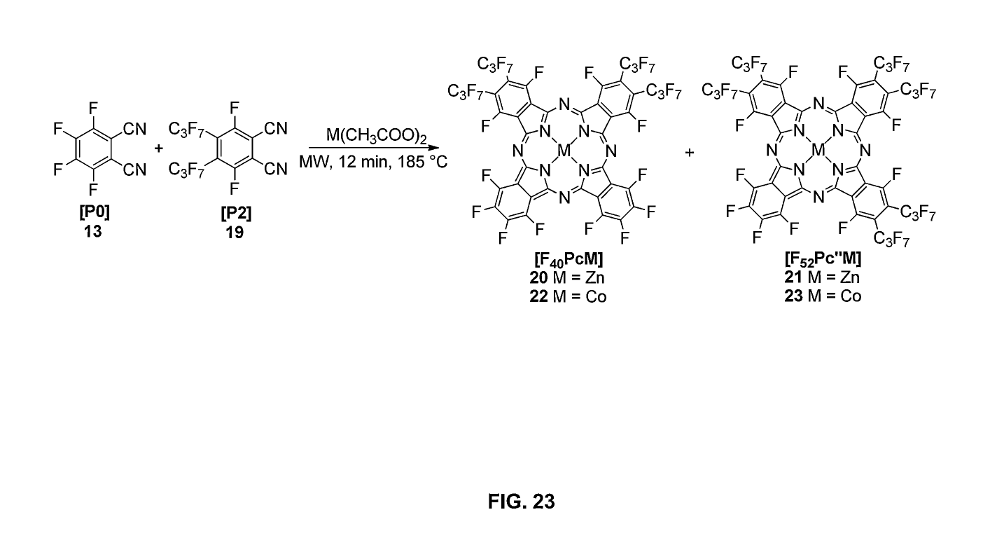

With reference to FIG. 23, an exemplary synthesis scheme for production of asymmetric F₄₀PcM and F₅₂Pc″M is depicted, showing the results of the combination of precursors P0 and P2 and including Compounds 20, 21, 22 and 23, which will be discussed in greater detail below. It should be noted that the F₄₀PcM and F₅₂Pc″M compounds are obtained together. Further, the approximately 2:2 molecular tetramerization of the two precursors yields a F₄₀PcM compound, while the approximately 1:3 tetramerization yields a F₅₂Pc″M compound. It should be noted that the Pc″ notation is used to differentiate the two F₅₂Pc compositional isomers (see, e.g., FIGS. 3A-E). In one experimental embodiment of this class of molecules the metal used is Co. As would be apparent to one of ordinary skill in the art, the present embodiment embraces the use of multiple other metals as the synthesis scheme is not metal specific and would include, but not be limited to, other metals with ionic radii that would be coordinated by the four nitrogen atoms of the phthalocyanines, e.g., Zn, Fe, Mg, Cu, and the like.

### Compounds 20 and 21

With reference to Compounds 20 and 21, an exemplary synthesis and characterization of F₄₀PcZn (hereinafter “Compound 20”) and F₅₂Pc″Zn (hereinafter “Compound 21”) is depicted. In particular, about twenty-five (25) 10 mL glass reaction vessels are charged each with about 0.4 g (about 0.79 mmol) perfluoro-4,5-diisopropyl phthalonitrile (depicted in FIG. 23 as P2 and hereinafter “Compound 19”), about 0.05 g (0.2 mmol) tetrafluorophthalonitrile (depicted in FIG. 23 as P0 and hereinafter “Compound 13”) and about 0.03 g (about 0.19 mmol) zinc(II) acetate dihydrate. After the addition of catalytic amounts of ammonium molibdate and about 1 mL nitrobenzene in each vessel, the vessels are sealed and heated in a microwave reactor at approximately 185° C. for about 12 min. The crude solid of each vial is extracted with about 25 mL ethyl acetate, the organic fractions are combined, concentrated in vacuo and adsorbed to silica gel (mesh size approximately 70-230). Chromatographic separation of the products is performed using neat hexane and then acetonehexane approximately 2:98 mixture (v/v), which allows for complete removal of nitrobenzene, unreacted Compound 19 and yellowish impurities. Then, a blue fraction consisting of exemplary F₆₄PcZn as a side product is eluted with an acetonehexane approximately 1:9 mixture, followed by a greenish-blue exemplary F₅₂Pc″Zn fraction (impurified with exemplary F₆₄PcZn). Finally, exemplary F₄₀PcZn is eluted with an acetonehexane approximately 2:8 mixture. Removal of the solvent under reduced pressure and re-purification of the products by gel filtration, evaporation and drying to constant weight allows for isolation of exemplary F₅₂Pc″Zn in about 25% yield (about 2.8 g) based on Compound 13, exemplary F₄₀PcZn in about 22% yield (about 1.1 g) based on Compound 13 and exemplary F₆₄PcZn in about 31% yield (about 3.2 g) based on Compound 19.

Specifically, the exemplary properties of Compound 20, i.e., F₄₀PcZn, are as follows: Mp>300° C.; UV-vis (CHCl₃): λₘₐₓ (log ε) 692 (5.24), 683 (5.23), 662 (4.80), 619 (4.67), 372 (4.84) nm (L mol⁻¹ cm⁻¹); IR (KBr): 1522, 1489, 1456, 1283, 1250, 1170, 1149, 1099, 965, 730 cm⁻¹; ¹⁹F NMR (282 MHz, (CD₃)₂CO): δ −71.56 (24F, s, CF₃), −103.85 (4F, br, Ar—F), −137.46 to −140.21 (4F, m, Ar—F), −149.56 to −150.85 (4F, m, Ar—F), −164.33 to −166.06 (4F, m, CF); HRMS (APCI+): calcd. for [M+]⁺ (C₄₄HF₄₀N₈Zn)⁺ 1464.8971. found 1464.8965.

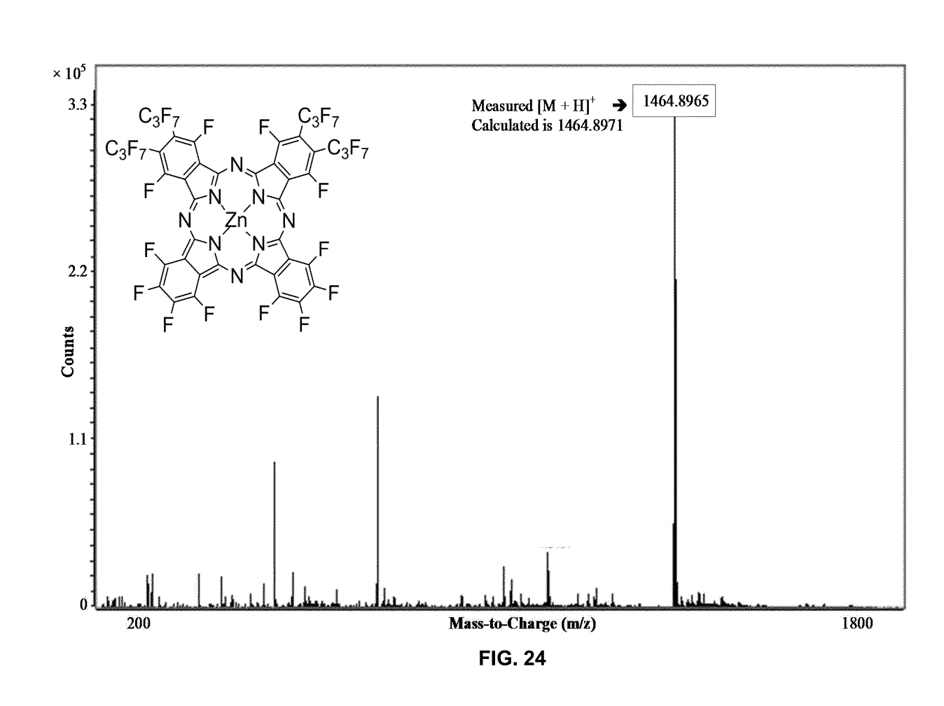

With reference to FIG. 24, the measured exact mass spectrum (positive ion APCI) and isotope pattern of [M+H]⁺ for F₄₀PcZn are depicted, indicating the calculated value for [M+H]⁺.

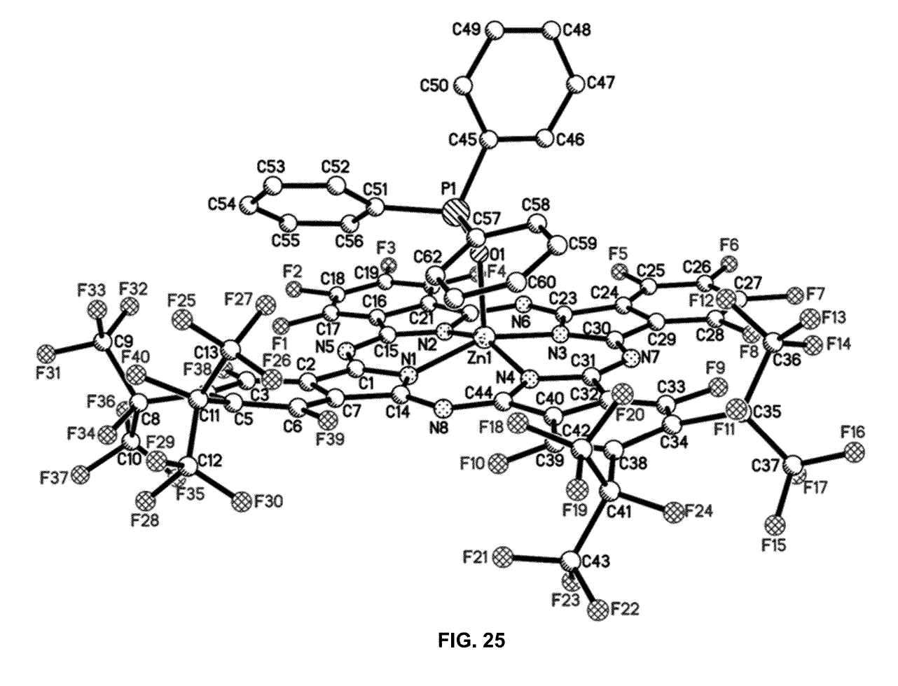

Turning now to FIG. 25, the X-ray structure of F₄₀PcZn(OPPh₃) is illustrated as a ball-and-stick representation, showing metal-coordinated triphenyl phosphine oxide with H atoms omitted.

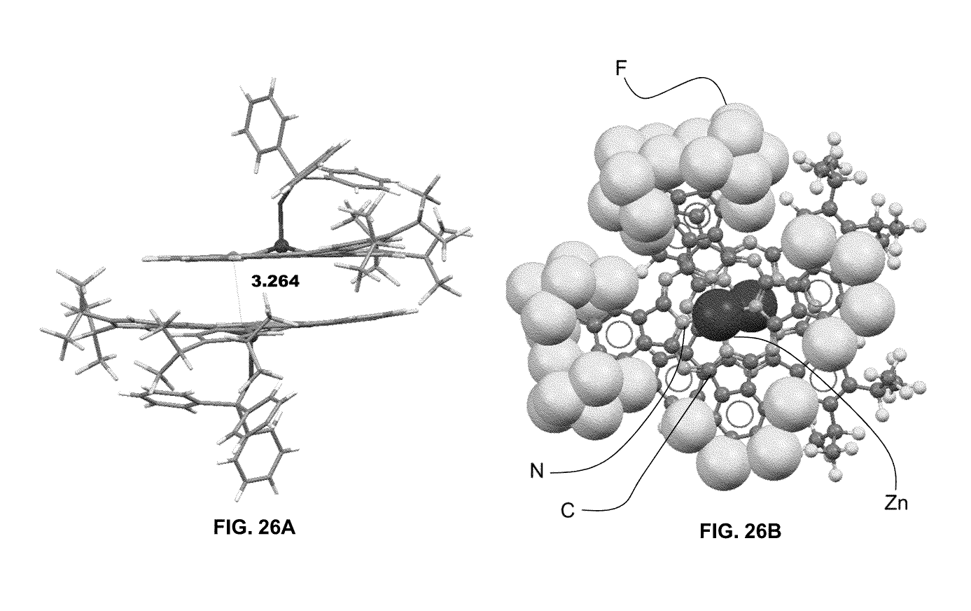

With reference to FIG. 26A, the side view of the aggregation in solid state of F₄₀PcZn(OPPh₃) is depicted, showing metal-coordinated triphenyl phosphine oxide. In particular, the toluene and chloroform molecules in the crystalline lattice have been omitted and the interplanar stacking distance, approximately 3.264 Å, demonstrates the existence of π-π interactions. With respect to FIG. 26B, a top-down view of the π-π stacking region of two adjacent molecules of F₄₀PcZn is illustrated. The F atoms of the i-C₃F₇ groups of the top molecule and the Zn atoms are specifically depicted as van der Waals spheres.

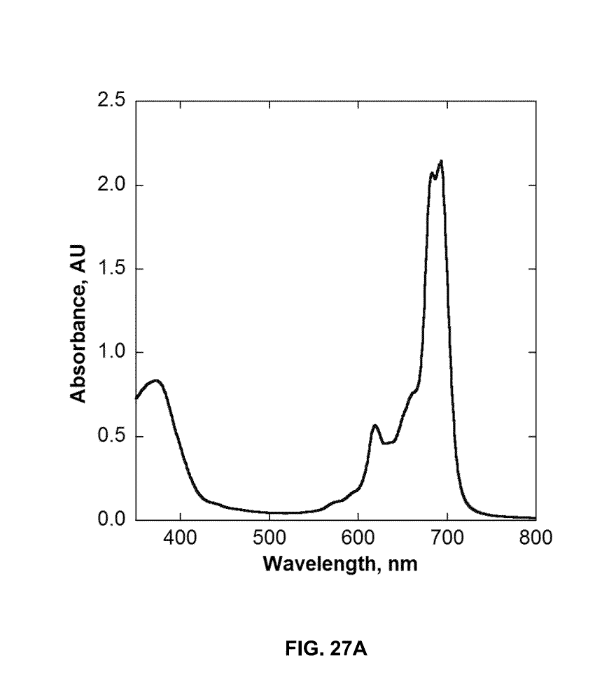

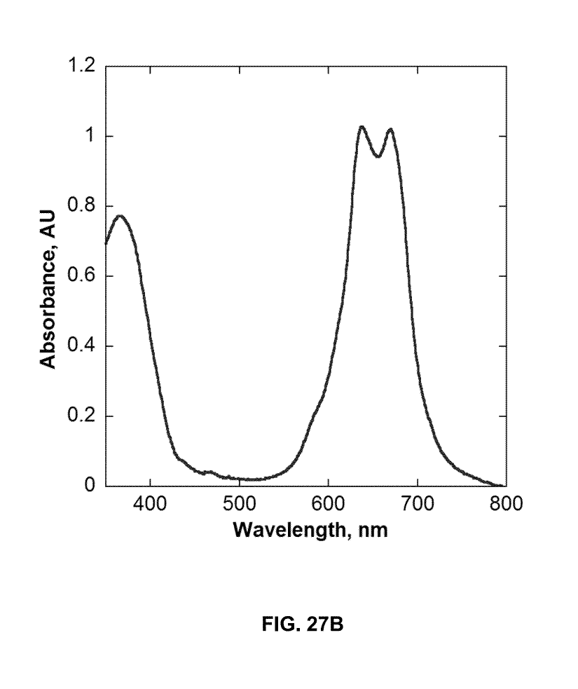

With reference to FIGS. 27A and B, the UV-Vis electronic absorption spectra of F₄₀PcZn are depicted, showing solvent-dependent aggregation. In particular, FIG. 27A illustrates a spectrum recorded in chloroform, in which F₄₀PcZn is a monomer, and FIG. 27B illustrates a spectrum recorded in ethanol, in which F₄₀PcZn displays strong aggregation.

Further, the exemplary properties of Compound 21, i.e., F₅₂Pc″Zn, are as follows: Mp>300° C.; UV-vis (CHCl₃): λₘₐₓ (log ε) 689 (5.00), 675 (4.97), 613 (4.34), 375 (4.50) nm (L mol⁻¹ cm⁻¹); HRMS (APCI+): calcd. for [M+]⁺ (C₅₀HF₅₂N₈Zn)⁺ 1764.8780. found 1764.8749.

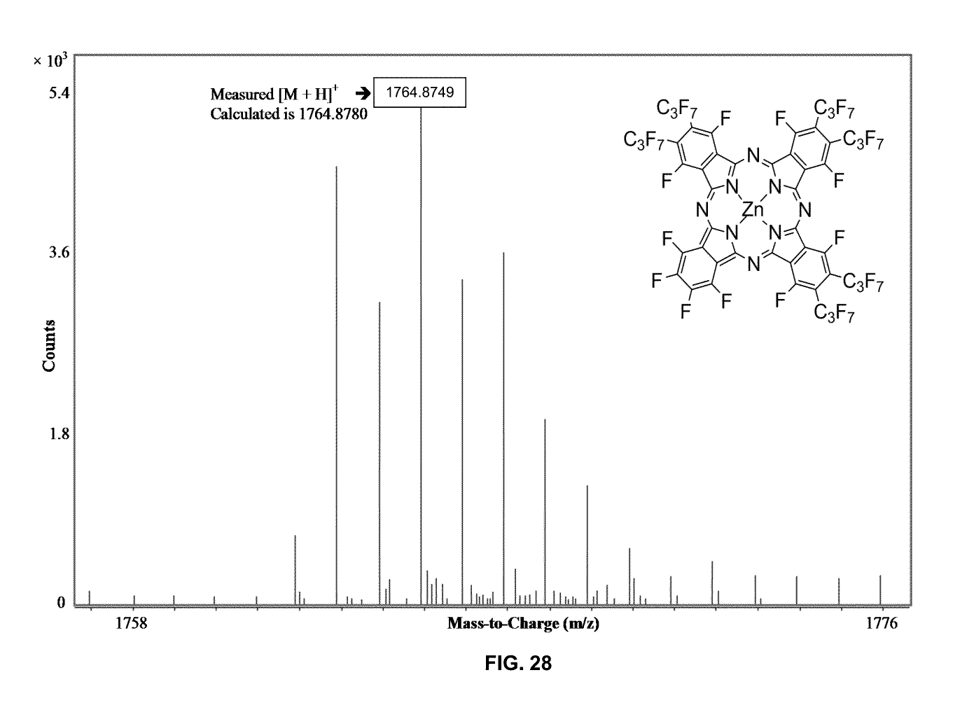

With reference to FIG. 28, the measured exact mass spectrum (positive ion APCI) and isotope pattern of [M+H]⁺ for F₅₂Pc″Zn are depicted, indicating the calculated value for [M+H]⁺.

### Compounds 22 and 23

With reference to Compounds 22 and 23, an exemplary synthesis and characterization of F₄₀PcCo (hereinafter “Compound 22”) and F₅₂Pc″Co (hereinafter “Compound 23”) is depicted. In particular, Compounds 22 and 23 are prepared similarly to Compounds 20 and 21, using ten (10) glass vessels, each charged with about 0.4 g (about 0.79 mmol) of Compound 19, about 0.05 g (about 0.25 mmol) of Compound 13 and about 0.05 g (about 0.19 mmol) cobalt(II) acetate tetrahydrate. Removal of nitrobenzene and unreacted precursor Compound 19 is performed by flash chromatography with hexane and then toluenehexane approximately 1:1 mixture (v/v). Exemplary F₆₄PcCo (side product) is eluted first, with an acetonehexane approximately 1:10 mixture, followed by royal blue exemplary F₄₀PcCo (acetonehexane 1:5) and finally dark green exemplary F₅₂Pc″Co, eluted with neat acetone. Repurification of Compounds 22 and 23 by flash chromatography with acetonehexane mixtures of gradually increasing polarity, followed by evaporation of the collected fractions and drying to constant weight allows for the isolation of exemplary F₄₀PcCo (Compound 22) in about 11% yield (about 0.22 g) and exemplary F₅₂Pc″Co (Compound 23) in about 0.3% yield (about 0.01 g), based on Compound 13. Exemplary F₆₄PcCo is isolated as a side product in about 18% yield (about 0.73 g) based on Compound 19. X-ray quality single crystals of exemplary F₄₀PcCo are obtained by slow evaporation of an acetone solution.

Specifically, the exemplary properties of Compound 22, i.e., F₄₀PcCo, are as follows: Mp>300° C.; UV-vis (CHCl₃): λₘₐₓ (log ε) 672 (4.88), 607 (4.28), 352 (4.50) nm (L mol⁻¹ cm⁻¹); IR (KBr): 1528, 1480, 1251, 1170, 1104, 964, 730 cm⁻¹; ¹⁹F NMR (282 MHz, (CD₃)₂CO): δ −71.38 (24F, s, CF₃), −104.56 (4F, br, Ar—F), −141.0 to −144.0 (4F, br, Ar—F), −154.0 to −158.0 (4F, br, Ar—F), −165.18 (4F, s, CF); HRMS (ESI+): calcd. for [M+H]⁺ (C₄₄HF₄₀N₈Co)⁺ 1458.8934. found 1458.8897.

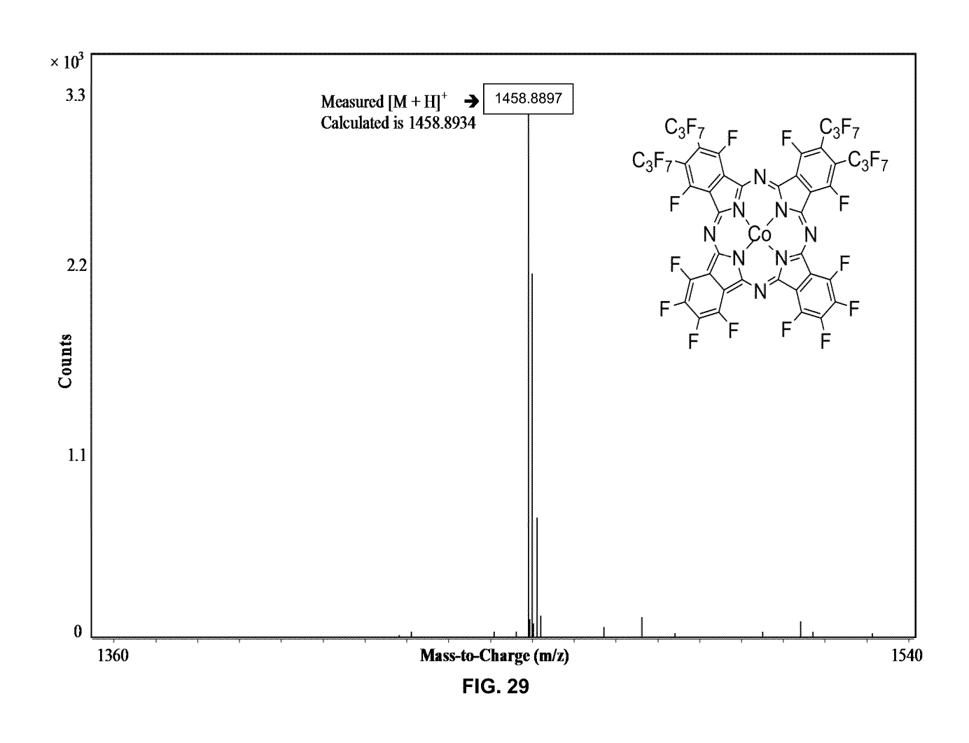

With reference to FIG. 29, the measured exact mass spectrum (positive ion ESI) and isotope pattern of [M+H]⁺ for F₄₀PcCo are depicted, indicating the calculated value for [M+H]⁺.

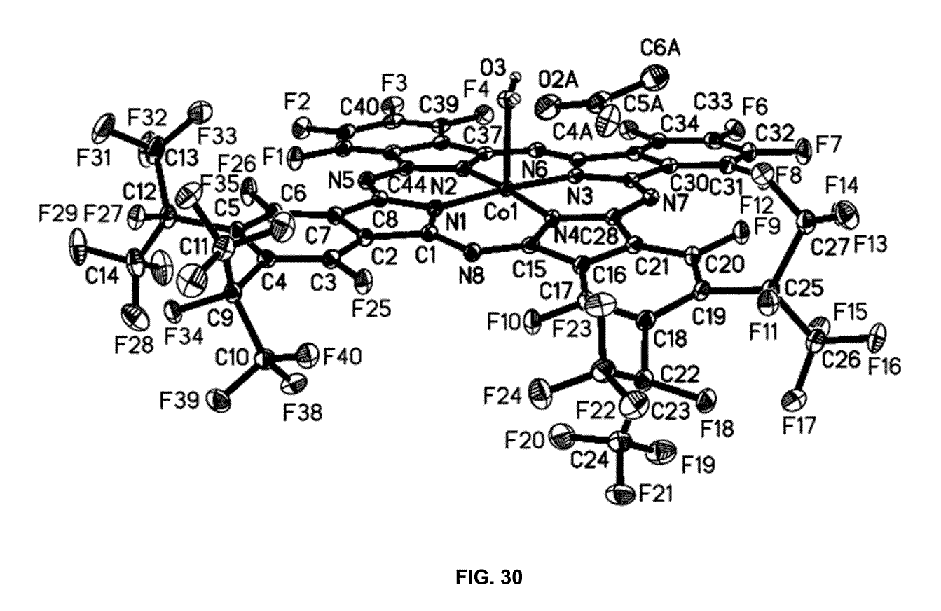

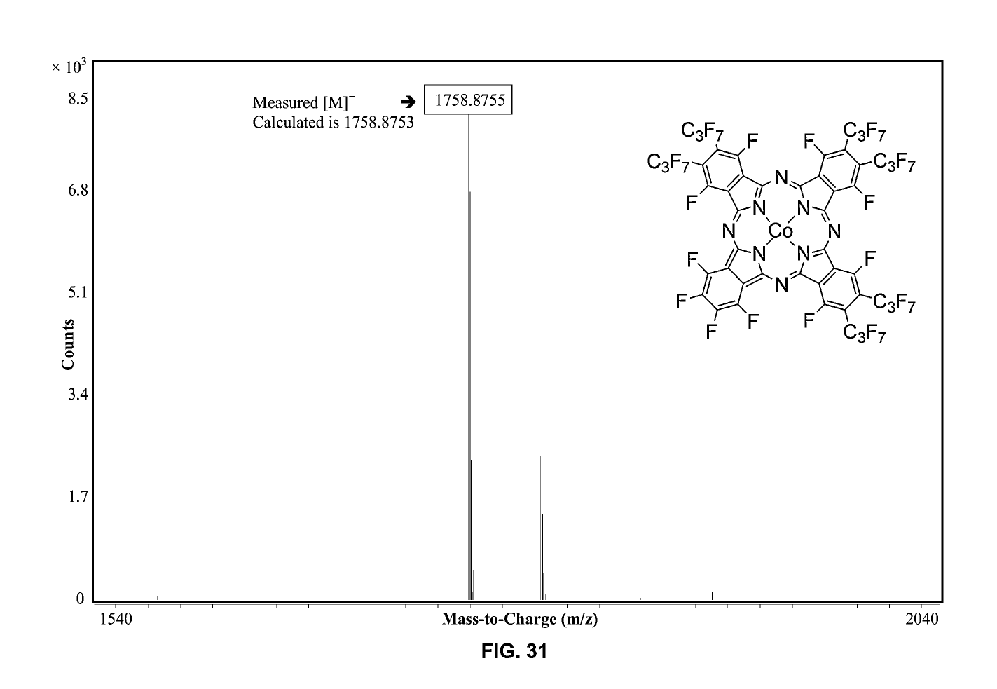

Turning now to FIG. 30, the X-ray structure of F₄₀PcCo(H₂O) is illustrated, showing metal-coordinated water and acetone molecules in the lattice. It should be noted that the thermal ellipsoids depicted are plotted at about 40% probability.

Further, the exemplary properties of Compound 23, i.e., F₅₂Pc″Co, are as follows: Mp>300° C.; UV-vis (CHCl₃): λₘₐₓ (log ε) 674 (3.94), 641 (3.86), 442 (3.85), 417 (3.84) nm (L mol⁻¹ cm⁻¹); ¹⁹F NMR (282 MHz, (CD₃)₂CO): δ −71.57 (36F, s, CF₃), −105.39 (6F, br, Ar—F), −137.0 to −143.0 (2F, br, Ar—F), −148.0 to −155.0 (2F, br, Ar—F), −165.17 (6F, s, CF); HRMS (APCI−): calcd. for [M]⁻ (C₅₀F₅₂N₈Co)⁻ 1758.8753. found 1758.8755.

With reference to FIG. 31, the measured exact mass spectrum (negative ion APCI) and isotope pattern of [M]⁺ for F₅₂Pc″Co are depicted, indicating the calculated value for [M]⁻.

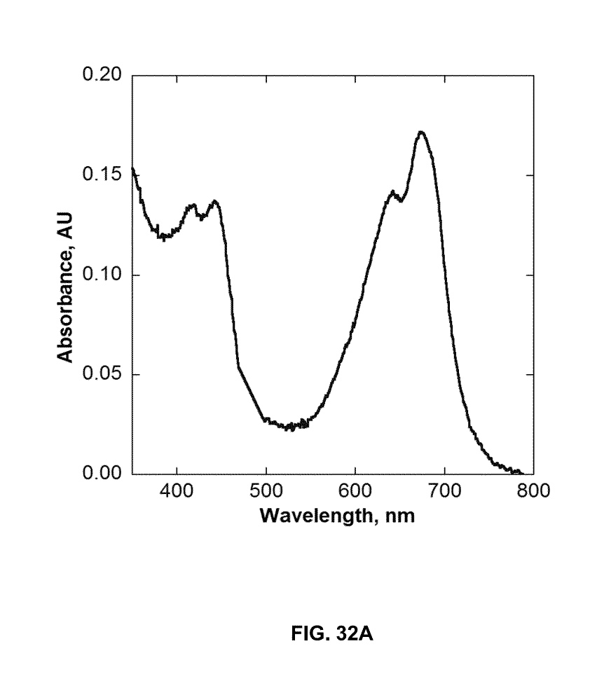

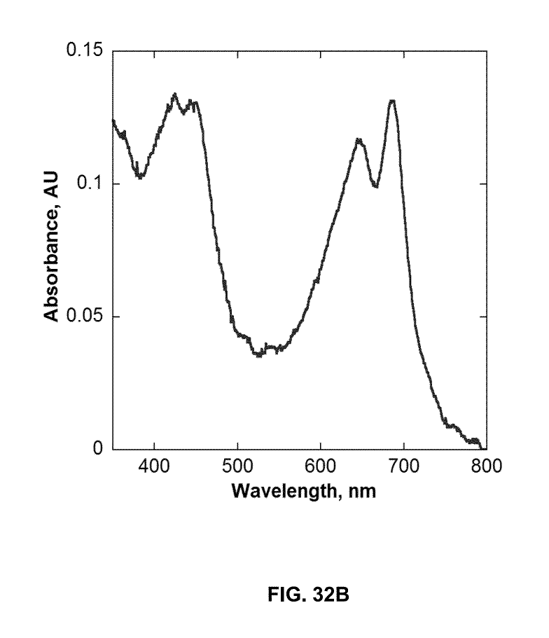

Turning now to FIGS. 32A and B, the UV-vis electronic absorption spectra of F₅₂Pc″Co is depicted, showing solvent-dependent aggregation. In particular, FIG. 32A illustrates a spectrum recorded in chloroform, in which F₅₂Pc″Co is slightly aggregated, and FIG. 32B illustrates a spectrum recorded in tetrahydrofuran, in which F₅₂Pc″Co shows an increased degree of aggregation.

### Catalytic Driven Pathway for Oxidizing Thiols

In accordance with embodiments of the present disclosure, novel catalytic driven pathways for oxidizing thiols are provided. In particular, the catalytic driven pathway for oxidizing thiols includes an iso-perfluoropropyl phthalocyanine catalyst and a redox reaction discussed with respect to Equations 1(a) and 1(b) below. The iso-perfluoropropyl phthalocyanine is generally F₆₄PcM and provides advantageous properties, including at least one of enhanced Pc solubility, production of X-ray quality crystals of a halogenated Pc, and depression of Pc frontier orbitals.

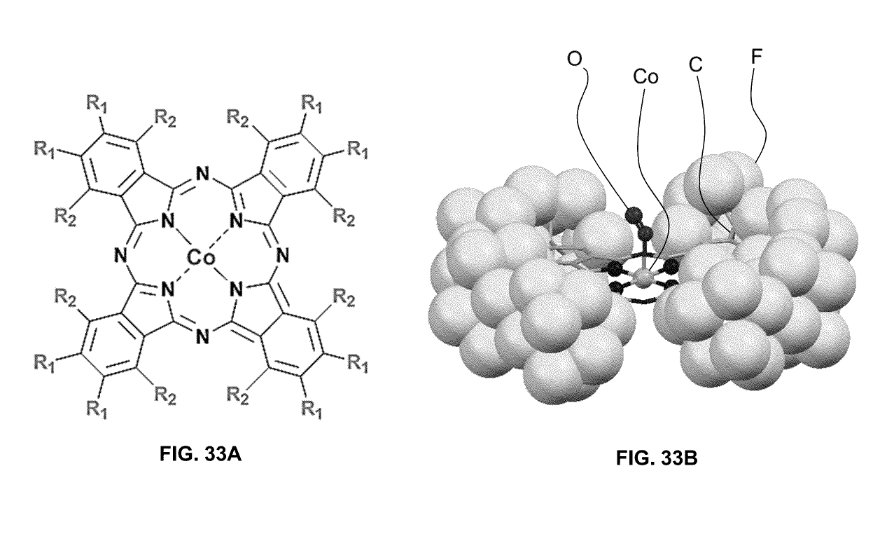

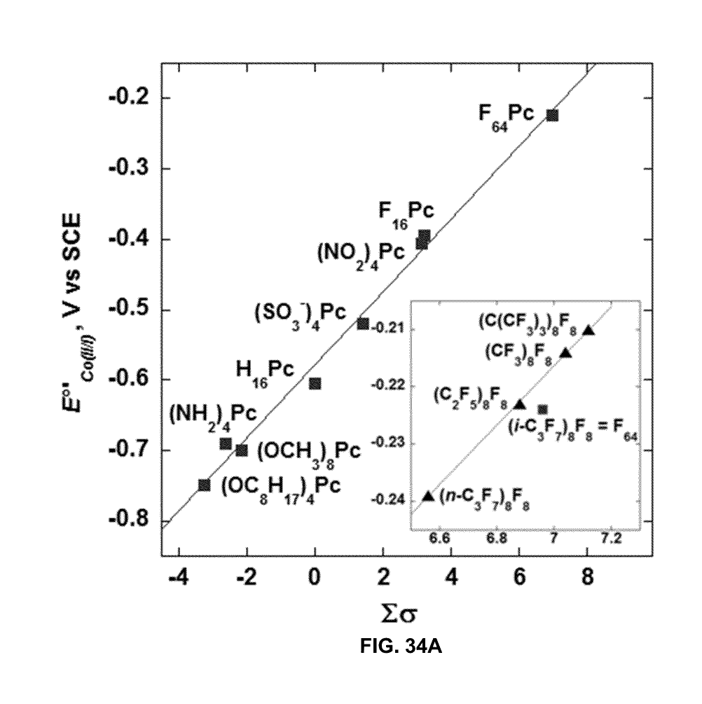

Organic-based molecules are problematic for aerobic oxidations since their C—H bonds are susceptible to radical attack. With reference to FIG. 33A, a general structure of exemplary cobalt phthalocyanines is illustrated. In particular, FIG. 33A illustrates compounds H₁₆PcCo (hereinafter “1-Co”), wherein R₁═R₂═H, F₁₆PcCo (hereinafter “2-Co”), wherein R₁═R₂═F, and F₆₄PcCo (hereinafter “3-Co”), wherein R₁−i-C₃F₇, R₂═F. FIG. 33B illustrates F₆₄PcCo(O₂) reaction intermediates, wherein O₂ stands for both O₂⁻ and O₂²⁻, drawn based on the X-ray structure of F₆₄PcCo.((CH₃)₂CO)₂ with the F groups shown as van der Waals spheres and the Co coordination sphere depicted as balls-and-sticks. It should be noted that the atomic coordinates of all atoms, except O₂, have been determined experimentally.

Still with reference to FIG. 33, in the case of metal phthalocyanines, e.g., H₁₆PcM (1-M), Cythochrome P450 related molecules, their C—H bonds and π-π stacking limit their utility as oxidation catalysts. The replacement of H by F to give F₁₆PcM (2-M) generally enhances Pc stability, eliminates electrophilic degradation, but favors nucleophilic susceptibility (see, e.g., Leznoff, C. C. et al., Chem. Comm., 338, (2004)) while promoting aggregation. Thus, even the strongest C—X bonds are typically insufficient to render this class of advantageous molecules completely stable. Replacing half of the F atoms of 2-M with iso-perfluoropropyl (i-C₃F₇) groups gives (i-C₃F₇)₈F₈PcM, abbreviated as F₆₄PcM (3-M), which results in advantageous properties, e.g., enhances Pc solubility, produces the first X-ray quality crystals of a halogenated Pc and depresses the Pc frontier orbitals (see, e.g., Bench, B. A. et al., Angew. Chem. Int. Ed., 41, 747 (2002), Bench, B. A. et al., Angew. Chem. Int. Ed., 41, 750 (2002); and Keizer, S. P. et al., J. Am. Chem. Soc., 125, 7067 (2003)). For 3-M, π-π stacking is disfavored both in solution and in the solid-state (see, e.g., Gerdes, R. et al., Dalton Trans., 1098 (2009) and Moons, H. et al., Inorg. Chem., 49, 8779 (2010)). Diamagnetic 3-Zn catalyzes the transfer of solar energy to ³O₂ to form ¹O₂ that oxygenates quantitatively an external substrate, (S)-(−)-citronellol (see, e.g., Keil, C. et al., Thin Solid Films, 517, 4379 (2009)).

Radical chemistry represents a challenge, which has been approached by examining a model reaction by the catalyzed autooxidation of corrosive and foul smelling RSH, a process generally practiced industrially (MEROX), catalyzed by partly sulfonated 1-Co (see, e.g., Basu, B. et al., Catal. Rev., 35, 571 (1993)). The overall reaction stoichiometry may be shown by 4 RSH+O₂→2 RSSR+2H₂O. Redox reaction pathways, via both Co(II)Co(III) and Co(II)Co(I) pairs are generally possible. In both cases S- and O-centered radicals are intermediates. For the relevant Co(II)Co(I) pathway, shown below, the coordination of RS⁻ to Co(II) is followed by (i) the reduction of Co(II) to Co(I) and formation of RS., (ii) oxidation of Co(I) by coordinated O₂ to regenerate Co(II) and form O₂^{.-}, i.e., superoxide. The cycle may be repeated to form O₂²⁻, i.e., peroxide, and RS. (see, e.g., Leung, P.-S. K. et al., J. Phys. Chem., 93, 430 (1989), Navid, A. et al., J. Porphyrins Phthalocyaninees, 3, 654 (1999), Schneider, G. et al., Photochem. Photobiol., 60, 333 (1994) and van Welzen, J. et al., Makromol. Chem., 190, 2477 (1989)). Reaction details may be shown in Equations 1(a) and 1(b):

RS⁻+PcCo(II)→[RS⁻—Co(II)Pc]→[RS.—Co(I)Pc] (1(a))

[RS.—Co(I)Pc]→RS.+PcCo(II)+e⁻ (1(b))

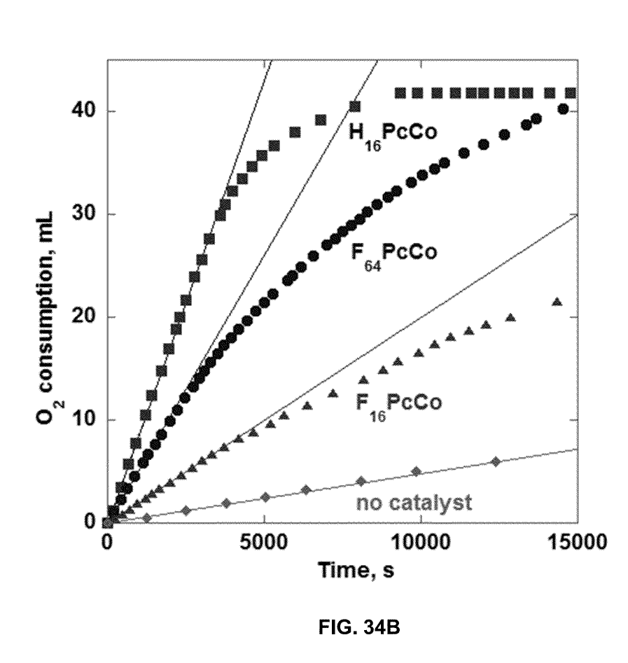

Soluble (SO₃H, SO₃Na)₄PcCo, and (COOH)_{2,4,8}PcCo (see, e.g., Shirai, H. et al., J. Phys. Chem., 95, 417 (1991) and Tyapochkin, E. M. et al., J. Porphyrins Phthalocyanines, 5, 405 (2001)) have been used to reveal mechanistic details in solution. Heterogenized systems used 1-Co, (COOH)₄PcCo, (NO₂)₄PcCo (see, e.g., Fischer, H. et al., Langmuir, 8, 2720 (1992)), (NH₂)₄PcCo (see, e.g., Buck, T. et al., J. Mol. Catal., 70, 259 (1991)), (SO₃Na)_{1,2}PcCo (see, e.g., Leitao, A. et al., Chem. Eng. Sci., 44, 1245 (1989)), and (SO₃⁻)₄PcCo (see, e.g., Chatti, I. et al., Catal. Today, 75, 113 (2002)). Polymer composites have also been used (see, e.g., van Welzen, J. et al., Makromol. Chem., 188, 1923 (1987) and van Welzen, J. et al., Makromol. Chem., 189, 587 (1988)). From a steric point of view, site-isolation in a matrix hinders the reaction of PcCoO₂ with another PcCo to form an inert μ-peroxo complex (see, e.g., Schutten, G. H. et al., Makromol. Chem., 180, 2341 (1979)). Turnover numbers generally increase, for example, for C₁₀H₂₁SH from about 150 to about 770 (see, e.g., Perez-Bernal, M. E. et al., Catal. Lett., 11, 55 (1991)). From an electronic point of view, since the Co(II) to Co(I) reduction is the rate determining step (r.d.s.), stabilization of Co(I) is desired. Overstabilization, however, could hinder catalyst reoxidation to Co(II), as depicted by Equation 1(b), and thus the catalytic process. Indeed, a Sabatier (volcano) plot of the rate of electrocatalytic oxidation of RSH vs. the PcCo(II)Co(I) reduction potentials exhibits a negative slope, indicating that the reoxidation to Co(II) generally controls the r.d.s. (see, e.g., Zagal, J. H. et al., Coord. Chem. Rev., 254, 2755 (2010) and Bedioui, F. et al., Phys. Chem. Chem. Phys., 9, 3383 (2007)). The potentials, in turn, correlate with substituents' Hammett constants, as illustrated in FIG. 34A.

In particular, FIG. 34A displays a plot of Pc(Co(II)Co(I)) reduction potentials vs. the sum of substituents' Hammett σ constants, wherein the following notation should be utilized: (SO₃⁻)₄Pc: R₁═SO₃⁻, H; (NH₂)₄Pc: R₁═NH₂, H; (NO₂)₄Pc: R₁═NO₂, H; (OCH₃)₈Pc: R₁═OCH₃; and (OC₈H₁₇)₄Pc: R₁═OC₈H₁₇, H (see, e.g., Zagal, J. H. et al., Coord. Chem. Rev., 254, 2755 (2010)). Further, the equation for the distribution may be depicted as y=−0.579+0.0518x, wherein the correlation coefficient is approximately 0.9955. With reference to the inset figure of FIG. 34A, the calculated reduction potentials for hypothetical (R_{f})₈F₈Pc, using R_{f} substituents with known Hammett constants, are illustrated (see, e.g., Hansch, C. et al., Chem. Rev., 91, 165 (1991)). Still with reference to the inset figure, the following notations should be utilized: R₂═F and R₁═R_{f} in ascending order of the E^{o}′_{Co(II/I)} potentials, i.e., propyl, isopropyl (F₆₄Pc, experimental point), ethyl, methyl, and t-butyl. Turning now to FIG. 34B, the O₂ consumption in the catalyzed autooxidation of 2-mercaptoethanol in aqueous tetrahydrofuran is illustrated.

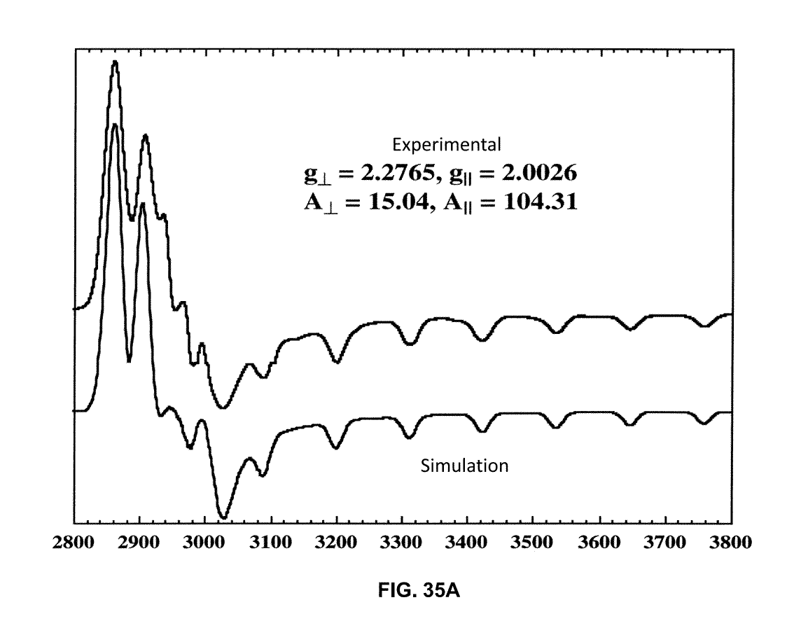

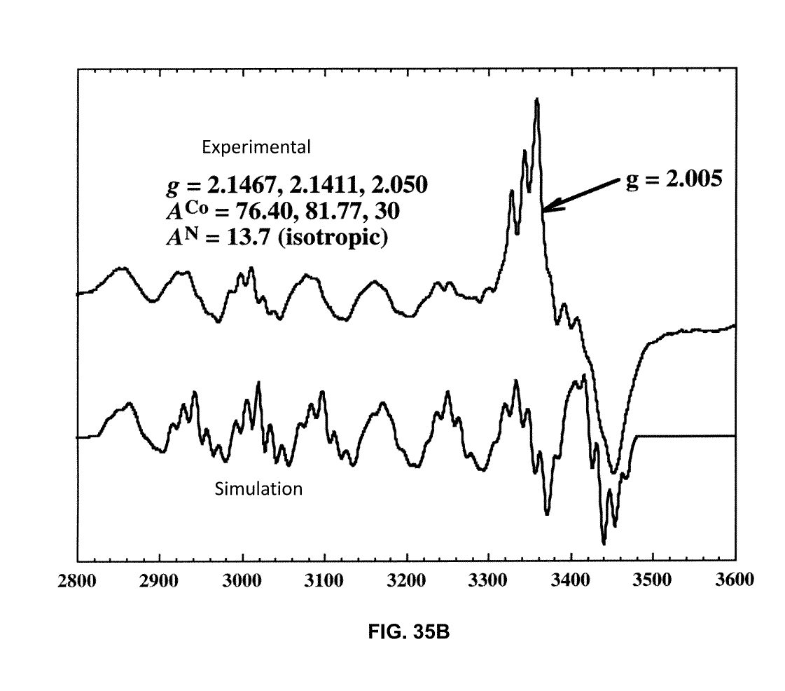

Previously, 2-Co was the extreme low-rate point due to the strongest F-induced stabilization of Co(I). The paramagnetic 3-Co, of certain exemplary embodiments of the present invention, may be electronically related to other PcCos, the majority exhibiting a singly occupied d_{z}² and equivalent d_{z} and d_{yz} orbitals (ESR in solution and solid-state, Table 1). Axial binding by the weakly coordinating acetone should be noted in solid-state. Coordination of N-methyl imidazole (ESR, FIGS. 35A and B) and ligand-independent site-isolation, e.g., for M=Zn (see, e.g., Gerdes, R. et al., Dalton Trans., 1098 (2009)), and Cu (see, e.g., Moons, H. et al., Inorg. Chem., 49, 8779 (2010)), in solution and in films (see, e.g., Keil, C. et al., Thin Solid Films, 517, 4379 (2009)) are characteristics imparted by the F₆₄Pc scaffold. The thermal stability of 3-Co is generally high and the complex sublimes in air at approximately 380° C. without decomposition. Interestingly, 3-Co cannot be electrochemically oxidized to Co(III) in DMF, but its reduction occurs at approximately E^{o}′=0.22 V (vs. SCE), thus justifying the choice of the Co(II)Co(I) catalytic pathway for certain embodiments of the present invention. Further, the Zn reduction value is about −0.30 V (see, e.g., Bench, B. A., Ph.D. Dissertation, Brown University (2001)).

TABLE 1
ESR parameters of selected phthalocyanines

| Complex | g_{⊥} | g_{∥} | Reference |
|---|---|---|---|
| H₁₆PcCo, | 2.60 | 1.99 | Cariati, F. et al., J. Chem. Soc., |
| in acetone | | | Dalton Trans., 556 (1975) |
| F₆₄PcCo, | 2.276 | 2.0026 | Loas, A. et al., Dalton Trans., |
| in acetone | | | 40, 5162 (2011) |
| F₆₄PcCo, | 2.282 | 2.0063 | Loas, A. et al., Dalton Trans., |
| powder | | | 40, 5162 (2011) |
| (SO₃H)₄PcCo, | 2.26 | 2.006 | Zwart, J. et al., J. Mol. Catal. |
| in DMF | | | 5, 51 (1979) |

A statistical X-ray analysis of all Co porphyrins (Por) and Pcs in the Cambridge Crystallographic Database (see, e.g., Allen, F. H., Acta Crystallogr. Sect. B, 58, 380 (2002)) indicates that Co deviates by less than about 0.1 Å from the ligand N₄ coordination plane regardless of its oxidation state (I, II or III) or coordination number. For Pcs, the mean CoN distances differ by approximately 1 e.s.d. when Co(II) and Co(III) are considered, i.e., approximately a 1.927±0.003 Å average. For the only PcCo(I) complex, the CoN distances range is approximately 1.879-1.914 Å with a mean of about 1.896 Å (see, e.g., Huckstadt, H. et al., Z. Anorg. Allg. Chem., 624, 715 (1998)). The shortening of the CoN distances upon reduction from Co(II) to Co(I), i.e., about 0.035 Å, is generally identical for both Por's and Pc's. It should be noted that the mean Co(II)N distance in 3-Co, i.e., about 1.926 Å, is typical for both Co(II) and Co(III) and thus Co(I) is not favored.

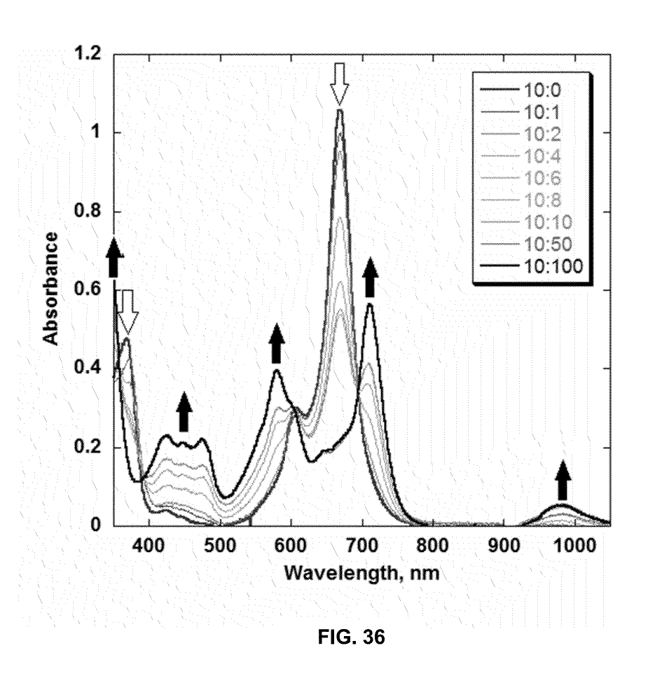

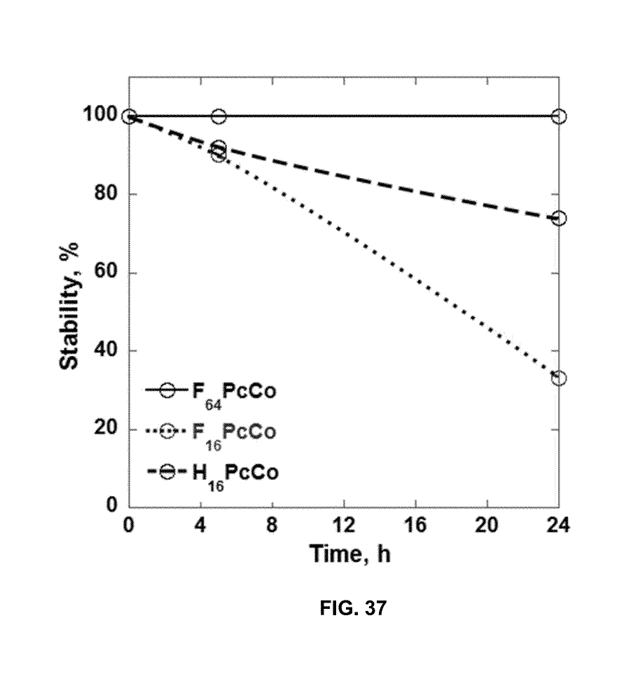

Taken together, the X-ray data suggests neither a structural hindrance for oxidation of Co(II) to Co(III), nor a preference for the reduction of Co(II) to Co(I). Thus, the 3-Co's record electronic deficiency, as shown in FIG. 34A, beyond 2-Co, is determined by electronic factors, e.g., aromatic F replacement by R_{f} groups exacerbates electronic deficiency due to loss of aromatic F π-back bonding. Relevant for catalysis, as illustrated by Equation 1(a) above, the reversible chemical reduction 3-Co(II)→3-Co(I) occurs in the presence of HO⁻ ions, as indicated by isosbestic points and the increase of the approximately 710 nm Q-band of the Co(I) complex at the expense of the approximately 670 nm Q-band of the Co(II) one (see FIG. 36). Further, addition of HCl completely reverses the reduction. In contrast, the isostructural F₆₄Pc(2-)Zn(II)→F₆₄Pc(3-)Zn(II) reduction is ligand centered. The actual catalytic activity of 3-Co is far from certain given (i) the inverse correlation between electron deficiency and thiol oxidation rates, (ii) strong S—Co bonds, a soft-soft type interaction and (iii) a high affinity for axial ligands. Thus, DFT frontier orbital energies calculations for 1-Co, 2-Co and (C₂F₅)₈F₈PcCo (F₄₈PcCo, 3′-Co) a surrogate for 3-Co, which is too large for the calculations, reveal that the ionization potentials increase by approximately 1.3 eV and approximately 1.1 eV from 1-Co to 2-Co and 2-Co to 3′-Co, respectively. Since C₂F₅ and i-C₃F₇ have similar Hammett constants (see, e.g., Hansch, C. et al., Chem. Rev., 91, 165 (1991)), illustrated by the inset of FIG. 34A, 3-Co and 3′-Co should have similar potentials. Electron affinity varies similarly, establishing progressively more difficult oxidationeasier reduction and more favorable axial binding as the F content increases.

Turning now to FIG. 34B, the results of thiol coupling using 1-, 2- and 3-Co and 2-mercaptoethanol (hereinafter “2-ME”) are shown. In particular, the reactions produce only the expected 2-hydroxyethyl disulfide (identified by ¹H and ¹³C NMR). No other S-oxidized products are observed, thus allowing an approximately 4:1 direct correlation between the number of moles of thiol and O₂ consumed, respectively. In the presence of about a 1000 fold molar excess of thiol, but in the absence of a base, 3-Co(II) is generally not reduced. In contrast, the formation of the thiolate ion upon addition of NaOH, [thiol]/[NaOH]=approximately 110/1, results in instantaneous appearance of 3-Co(I) (as demonstrated by UV-Vis, FIG. 36). Immediate O₂ uptake occurs only when both RS⁻ and the catalyst are present. It is noted that light makes no difference indicating absence of solar energy transfer. With reference to Table 2, the catalysis parameters are listed below:

TABLE 2
Parameters of the catalyzed autooxidation of 2-mercaptoethanol

| Catalyst | Stability^{a} | Rate^{b} | TOF^{c} | TON^{d} |
|---|---|---|---|---|
| H₁₆PcCo | 75% | 23.8 | 3.0 | 12,600 |
| F₁₆PcCo | 35% | 4.9 | 0.84 | 7,700 |
| F₆₄PcCo | >99% | 12.8 | 1.74 | 13,000 |

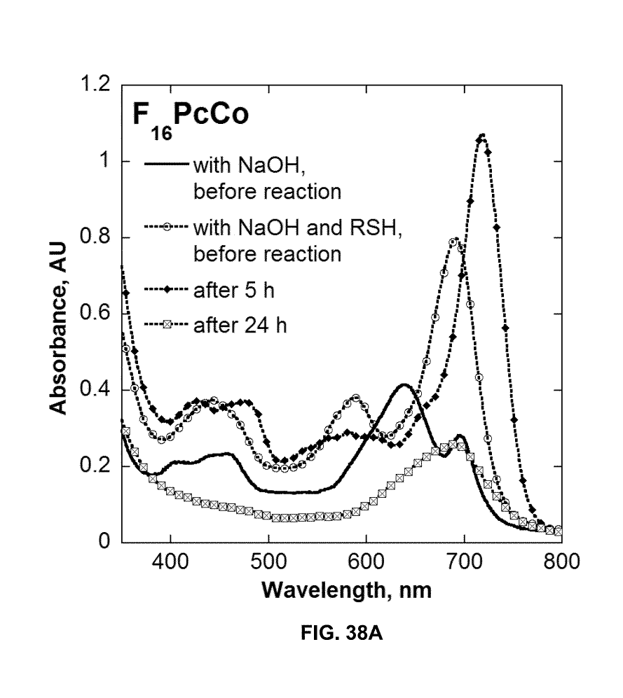

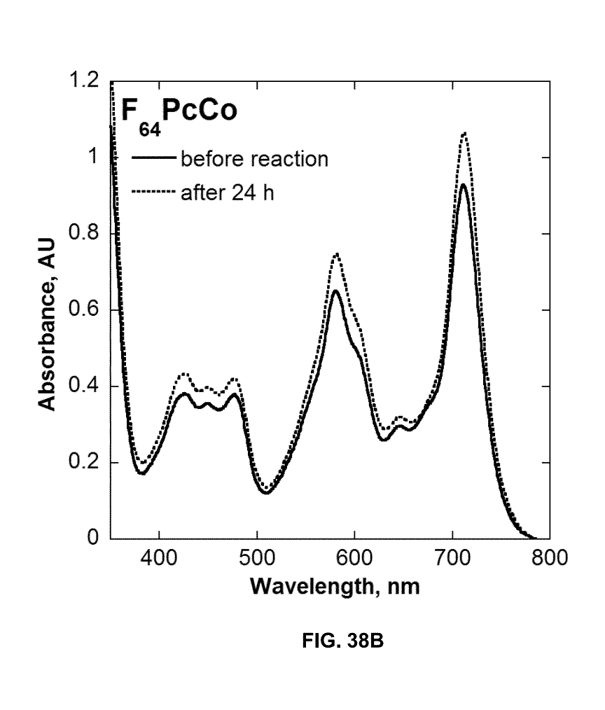

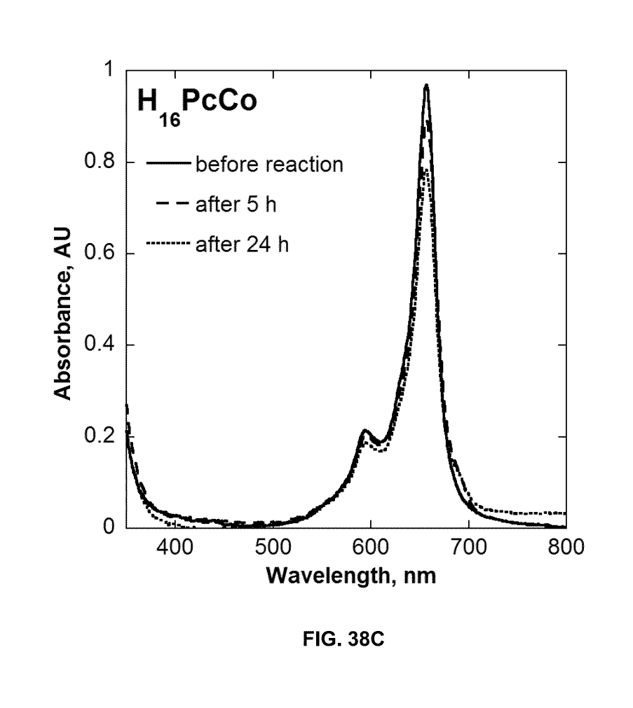

^{a}Stability is defined as the ration of (Q-band intensities after 24 hours/initial intensities) × 100. See also FIGS. 38A-C. Pc degradation products have not been identified.
^{b}Initial reaction rate, i.e., μmol O₂ min⁻¹, calculated from the linear fit portion of FIG. 34A.
^{c}Turnover frequency, i.e., RSH sec⁻¹ mol Pc⁻¹, calculated under pseudo-first order conditions.
^{d}Total oxidation number after 5 hours, limited by the RSH batch reaction to approximately 13,000.

3-Co is highly stable at about 25° C. under the reaction conditions with nucleophiles and radicals present. Moreover, 3-Co showed no degradation for at least two (2) days in refluxing, basic aqueous tetrahydrofuran, or concentrated H₂SO₄. Since the aromatic F substituents in 3-Co should generally be more susceptible to nucleophilic attack relative to 2-Co, the protective steric effect imparted by the i-C₃F₇ groups becomes apparent.

The initial oxidation rates are partly incongruent with the reduction potentials. In particular, the calculated ratio of initial reaction rates for 2-Co/1-Co based on reduction potentials is about 0.16 vs. the observed value of about 0.843.0=0.28. In contrast, 3-Co, presumably less efficient than 2-Co, has a rate approximately twice as high, about 20 times faster than predicted based on reduction potentials. Since the reoxidation of Co(I) to Co(II) (the r.d.s.) proceeded as expected based on free energy correlations, the discrepancy is unexplainable on electronic grounds alone. Potential reasons for the enhanced rate of 3-Co includes: (i) R_{f} steric crowding leading to an accelerated departure of the thiyl radical (product), a classical feature of enzymatic reactions and consistent with the limited miscibility of hydrocarbons and fluorinated solvents, (ii) an R_{f}-induced extra loss of Co²⁺ polarizability, making it unlikely to bind soft S-radicals, and (iii) hydrophobic preference for neutral (thiyl radical) over charged (thiolate) species in the immediate R_{f} catalytic environment. Steric crowding could destabilize [RS⁻—Co(II)Pc], which may exhibit an approximately 2.2 Å Co—S bond (see, e.g., Cardenas-Jiron, G. I. et al., J. Mol. Struct., 580, 193 (2002)), the sp³ hybridized S forcing the thiolate backbone too close to the R_{f} groups. This destabilization generally vanishes upon electron transfer and departure of the resulting thiyl radical. Thus, the results suggest that 3-Co appears to exhibit strong RS—Co binding, a potential “deficiency”, but which could be used to broaden its reactivity spectrum to include less basic thiols.

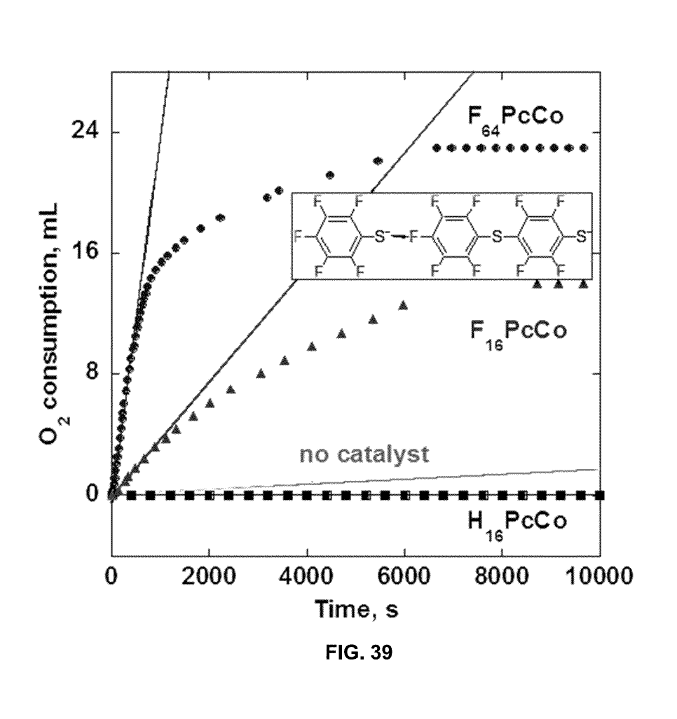

This use also provides an alternative exemplary thiol coupling. In particular, perfluoro benzenethiol (hereinafter “PBT”) is a poor nucleophile, at least one million times more acidic than 2-ME, their pKa values being about 2.68 and about 9.2, respectively (see, e.g., Martell, A. E. et al., Critical Stability Constants, vol. 3, Plenum Press, New York (1977)). Thus, the critical steps of thiolate coordination and electron transfers may not occur for PBT. Indeed, to the best of our knowledge, the aerobic coupling of PBT has not been reported. No oxidation was observed with 1-Co, unlike the case of 2-ME. In contrast, 3-Co produces PBT disulfide (identified by ¹⁹F NMR), approximately 6.4 times faster than 2-Co with an yield about 1.6 times as high, about 53% and about 32%, respectively (see FIG. 39). The low yields are due to a parallel, unrelated reaction of the PBT anion, C₆F₅S⁻, which dimerizes via nucleophilic attack to yield the thioether-thiol C₆F₅S-p-C₆F₄S⁻ (see, e.g., Namuswe, F. et al., J. Am. Chem. Soc., 130, 14189 (2008)). Further, glass corrosion was observed, potentially due to HF. Consequently, the PBT anion concentration decreases (¹⁹F NMR), consistently with the lower total O₂ uptake.

The extreme electronic deficiency of 3-Co is actually beneficial in securing efficient binding of an acidic thiol and subsequent electron transfer, events that typically do not occur with the parent 1-Co, or occur less efficiently with the sterically unhindered and electronically richer (relative to 3-Co) 2-Co.

Despite F₆₄Pc scaffold electronic deficiency, activation of O₂ generally occurs within the R_{f} pocket of 3-Co by two, one-electron transfer steps to form O₂^{.-} and O₂²⁻. The F₆₄Pc ligand is thus able to suppress electron loss from Co(II), but not from Co(I). The 1:1 F:R_{f} ratio appears suitable for both catalyst stability and activity in certain disclosed embodiments of the present invention. Its lowering might prevent electron loss even from the Co(I) level, thus stopping the catalysis, while its increase could lead to catalyst instability. Notably, the stepwise reduction of O₂ to O₂²⁻ without disproportionation is known for the N₄S(thiolate) chromophore of superoxide reductases (SOR), but with M=Fe. Strong trans thiolate binding is believed to weaken the M-O bond, thus favoring the release of H₂O₂ (see, e.g., Namuswe, F. et al., J. Am. Chem. Soc., 130, 14189 (2008)), an effect relevant to the present disclosure since H₂O₂ released from the Co center contributes to thiol coupling.

In summary, disclosed is a first member of a family of three-dimensional, metal-organic aerobic catalysts whose organic ligand framework is designed to stabilize it against all possible degradation pathways. Coordination and reduction of O₂ within a fluorinated active site pocket leads to both O- and S-centered radicals, the latter coupling to disulfides.

Further, the stabilization of ligand composition, while offering labile sites for catalysis, is also a challenge that responds to identified future technology needs (see, e.g., Lippard, S. J., Nature, 416, 587 (2002)). In particular, the fluoro-perfluoroalkyl substituents offer an answer within phthalocyanines and, maybe, other frameworks.

In one exemplary embodiment of the present invention we have a process in which the catalyst is a chemically robust phthalocyanine in which all C—H bonds of said molecule have been replaced by a combination of F and perfluoro-isopropyl groups and which displays a redox metal center with high Lewis acidity.

The properties of the phthalocyanines described above show how the industrial process of oxidative coupling of corrosive thiols to disulfides, i.e., petroleum sweetening, can be advantageously improved by the novel and highly-stable, yet active, catalyst class. Some potentially advantageous properties of the disclosed exemplary catalysts include, but are not limited to, e.g., lower need for catalyst replacement, spent catalyst separations, disposal cost, and the like.

Although the present disclosure has been described with reference to exemplary embodiments and implementations, it is to be understood that the present disclosure is neither limited by nor restricted to such exemplary embodiments and/or implementations. Rather, the present disclosure is susceptible to various modifications, enhancements and variations without departing from the spirit or scope of the present disclosure. Indeed, the present disclosure expressly encompasses such modifications, enhancements and variations as will be readily apparent to persons skilled in the art from the disclosure herein contained.

1. A method of oxidizing thiols in a catalytic driven pathway, comprising:
providing a catalyst, wherein the catalyst is an iso-perfluoropropyl phthalocyanine catalyst; and
conducting a redox reaction in the presence of the catalyst, wherein the redox reaction is shown by:
RS⁻+PcCo(II)→[RS⁻—Co(II)Pc]→[RS.—Co(I)Pc], (i)
and
[RS.—Co(I)Pc]→RS.+PcCo(II)+e⁻. (ii)

2. The method according to claim 1, wherein the iso-perfluoropropyl phthalocyanine catalyst is F₆₄PcM, and wherein P_{C} of F₆₄PcM represents a phthalocyanine and M of F₆₄PcM represents a metal.

3. The method according to claim 1, wherein the iso-perfluoropropyl phthalocyanine catalyst provides a higher P_{C} solubility, an increased production of X-ray quality crystals of a halogenated P_{C}, and a depression of P_{C} frontier orbitals, relative to a perfluorophthalocyanine.

4. The method according to claim 2, wherein the metal is selected from a group consisting of Zn, Co, Fe, Mg and Cu.

5. The method according to claim 1, wherein the iso-perfluoropropyl phthalocyanine catalyst provides increased P_{C} stability, decreased electrophilic degradation, improved nucleophilic susceptibility, and improved aggregation, relative to a perfluorophthalocyanine.

6. The method according to claim 1, wherein the iso-perfluoropropyl phthalocyanine exhibits an asymmetric orientation.

7. The method according to claim 1, wherein the iso-perfluoropropyl phthalocyanine exhibits tunable π-π stacking.
